(19) United States
(12) Patent Application Publication
(10) Pub. No.: US 2017/0184590 A1
(43) Pub. Date: Jun. 29, 2017

(54) DETECTION OF ANTIGENIC VARIANTS

(71) Applicant: XIUFENG WAN, Starkville, MS (US)

(72) Inventor: XIUFENG WAN, Starkville, MS (US)

(73) Assignee: Mississippi State University, Starkville, MS (US)

(21) Appl. No.: 15/390,396

(22) Filed: Dec. 23, 2016

Related U.S. Application Data
(60) Provisional application No. 62/387,165, filed on Dec. 23, 2015.

(51) Int. Cl. G01N 33/569; C12Q 1/68

(52) U.S. Cl. CPC G01N 33/56983; C12Q 1/686; C12Q 1/6804; G01N 33/56911

(57) ABSTRACT

An antigenic characterization method using polyclonal antibody-based proximity ligation assays (polyPLA). Methods, kits, and other tools disclosed herein are useful in detecting microbial antigenic variants in samples, including clinical samples. The methods and kits have great utility in detecting antigenic variants for pathogenic microbes, including viruses, bacteria, and parasites.

### CROSS-REFERENCE TO RELATED APPLICATIONS

This application claims priority to U.S. Provisional Application No. 62/387,165 to Xiufeng Wan filed on Dec. 23, 2015, the contents of which are incorporated herein by reference in its entirety.

### GOVERNMENT SUPPORT

This invention was made with government support under grant number 1RC1AI086830-01 awarded by the National Institute of Health. The government has certain rights in the invention.

### FIELD OF THE INVENTION

The disclosed subject matter generally relates to detection of antigenic variants of pathogens and methods, kits, and other tools for use in detecting antigenic variants of pathogens.

### BACKGROUND OF THE INVENTION

Unless otherwise indicated herein, the approaches described in this section are not prior art to the claims of this invention, and are not admitted to be prior art by inclusion herein.

Pathogens, such as viral and bacterial pathogens, present a perpetual threat to public health, particularly as antigenic variations of pathogens are generated and/or as antigenic drift occurs. The influenza virus, for example, is associated with thousands of deaths every year in the United States (2010). A worldwide pandemic could increase the death toll to millions in a short period of time. The hallmark of the influenza virus is antigenic variation, which comes in two forms: antigenic drift and antigenic shift; leading to the recurrence of influenza virus infections (Katz et al., 1987). Mutations in the hemagglutinin and neuraminidase glycoproteins cause antigenic drift. Meanwhile, antigenic shift is caused by the replacement of a new subtype of hemagglutinin and sometimes neuraminidase through genetic reassortment.

The influenza vaccine is the most viable option in counteracting and reducing the impact of influenza outbreaks (Harper et al., 2004). Since influenza viruses are continuously changing their antigenicity in order to escape the host immunity (Nobusawa and Nakajima, 1988; Webster et al., 1982), the vaccine strains need to be updated almost annually to obtain antigenic matches between the vaccine strain and the strain potentially causing future outbreaks (Ampofo et al., 2012; Gerdil, 2003). Identification of influenza antigenic variants is the key to a successful influenza vaccination program for both pandemic preparedness as well as seasonal influenza prevention and control (Katz et al., 1987).

Routinely, immunological tests, such as hemagglutination inhibition (HI) assays and microneutralization (MN) assays, have been relied upon to identify antigenic variants among the circulating strains (Medeiros et al., 2001). The HI assay is an experiment to measure how a test influenza antigen and a reference antigen (e.g., a current vaccine strain) match through the immunological reaction between the test antigen and the reference antiserum. This reference antiserum is usually generated in animals (i.e., ferrets) using the reference antigen or collected from human subjects. HI assays are limited due to their use of red blood cells (RBCs), e.g., turkey red blood cells, as indicators for the binding affinity of antigen and antiserum (Services, 1982). A higher interaction between antigen and antisera will lead to less hemagglutination of RBCs (Hirst, 1941). Compared to HI assays, MN assays seem to be more sensitive and specific but are much more time-consuming. Moreover, for influenza viruses requiring biosafety-level 3 (BSL-3) or higher, MN assays are difficult to perform (Grund et al., 2011). For this reason, HI assays have been one of the routine procedures used to identify influenza antigenic variants for vaccine strain selection while MN assays are generally used to validate the results from HI assays.

However, the data from HI assays are notoriously noisy, and HI experiments are affected by many factors. For example, RBCs used from different species and even variation in RBC sialic acid receptors can produce varied results (Medeiros et al., 2001). The data are subjective interpretations and the HI assays have difficulty in automating and standardizing operations. Minor antigenic variants within a heterogeneous population cannot be assessed by the serological method of HI (Patterson and Oxford, 1986). More importantly, mutations of the receptor binding site in HA (Nobusawa and Nakajima, 1988) (antigenic drift) are causing human seasonal H1N1 (Azzi et al., 1993; Morishita et al., 1993) and H3N2 influenza A viruses (Nobusawa et al., 2000) to lose the ability to bind to certain types of RBCs. For example, the mutations at residue 193, 196, 197, and 225 in the human epidemic H1N1 influenza A viruses in 1988 or later caused the loss of their abilities to agglutinate chicken RBCs due to four amino acid changes (Morishita et al., 1996). For H3N2 viruses, the Gly190Asp substitution has been correlated to the loss of the ability to agglutinate chicken erythrocytes (Cox, 1995; Fitch et al., 1997; Lindstrom et al., 1998; Lindstrom et al., 1996; Medeiros et al., 2001; Mori et al., 1999; Nobusawa et al., 2000). Since 2000, human seasonal H1N1 and H3N2 influenza A viruses have been losing their binding abilities to turkey red blood cells (Medeiros et al., 2001; Oh et al., 2008). This may be attributed to a reduced affinity for sialic acid-linked receptors (particularly α2-6-linked receptors), which are at lower levels on chicken and turkey RBC compared to levels on guinea pig RBC (Medeiros et al., 2001; Oh et al., 2008). Consequently, a critical demand exists for the development of a red blood cell independent assay for influenza antigenic variation.

Proximity ligation assay utilizes quantitative PCR (qRT-PCR) for the detection of antigen-antibody interaction (Schlingemann et al., 2010). For this assay: (1) oligonucleotide-linked monoclonal antibodies are incubated with the analyte in question; (2) if in close proximity, the oligonucleotides can be ligated together; and (3) presence of analyte will be shown by amplification of ligated products with qRT-PCR. The assay reporter signal is dependent on a proximal and dual recognition of each target analyte providing high specificity (Fredriksson et al., 2007).

### SUMMARY OF THE INVENTION

In one aspect, the present invention provides unique antigenic characterization methods using a polyclonal antibody-based proximity ligation assay (polyPLA). The methods can be used to detect an antigenic variation in a variety of pathogen samples, including pathogenic bacteria and viruses. This method was found to be useful in detecting, for example, influenza antigenic variants in clinical samples. Thus, the methods can be used directly on clinical samples without the need for expensive and time-consuming pathogen sample propagation and purification. The methods can be used to detect antigenic variation for those microbes in clinical samples which are either cultivable or uncultivable in laboratory setting.

In another aspect, the present invention provides unique methods of detecting antigenic characteristics of an animal pathogen, including pathogenic bacteria, viruses, and other microbes. The detected antigenic characteristics can then be used to design vaccines for the animal pathogen. In some embodiments, the animal may be a human or a non-human animal susceptible to an infection by pathogens that experience antigenic variations. In some embodiments, the pathogen may be a bacterium, such as Escherichia coli, Campylobacter jejuni, Bordetella pertussis, Haemophilus influenzae, Streptococcus pneumoniae, Neisseria gonorrhoeae, and Neisseria meningitidis, a virus, such as influenza A and HIV, dengue virus, a parasitic protozoan, such as trypanosomiasis and malaria, or others. The foregoing are examples only, and in no way limiting of the utility of the present inventions disclosed herein.

In still another aspect, the present invention provides unique kits for detecting antigenic characteristics of a human or non-human pathogen, including pathogenic bacteria, viruses, and other microbes.

### BRIEF DESCRIPTION OF THE DRAWINGS

Further advantages of the invention will become apparent by reference to the detailed description of preferred embodiments when considered in conjunction with the drawings which form a portion of the disclosure and wherein:

FIGS. 1A-1G. The simplified diagram of polyPLA. polyPLA quantifies the antibody antigen binding avidity using the amplification signals in quantitative PCR (qPCR) from the pairs of primers attached to a reference polyclonal antiserum. First, polyPLA biotinylates a reference polyclonal antiserum (FIG. 1A), which will be then labeled with sodium azide-linked 5′ and 3′ oligonucleotides (FIG. 1B). A labeled polyclonal antiserum with ΔCt≧8.5 in the ligation efficiency test is then incubated with a reference antigen (virus) or a testing antigen (FIG. 1C), followed by the proximity ligation of the two oligos (FIG. 1D). The antibody antigen binding avidity is quantified using the amplification signals ΔCt in qPCR (FIG. 1E). The ΔCt values among the polyclonal antisera and antigens can be compared to assess antigenic differences among these tested antigens, and these ΔCt values can be viewed as similar to the serological titers, such as HI and neutralization titers, from conventional serological assays (FIG. 1F). The polyPLA units were normalized by its ΔCt values for polyclonal antiserum (polyΔCt) with its ΔCt values for monoclonal antibody against NP (monoΔCt) (FIG. 1G).

FIG. 2. Optimization of the methods in detecting NP proteins using proximity ligation assays. OV denotes the control viruses that were harvested directly after viral propagation in MDCK cells; F-T denotes the viruses that were frozen and thawed five times; L denotes the viruses that were treated with lysis buffer.

FIG. 3. PolyPLA detects predominant IgG against HA gene. The ΔCt of proximity ligation assays for SY/05(H3N2), PR8(H1N1), and three reassortants SY05xPR8(H3N2), SY05xPR8(H1N2), and SY05xPR8(H3N1) were measured using reference polyclonal sera against SY/05(H3N2).

FIG. 4. The linear correlation of polyΔCt (R=0.98) and monoΔCt (R=0.92) with viral quantities. The cutoff ΔCt value 3.00 was equivalent to 4.90×10⁴ TCID₅₀/mL against its homologous polyclonal antibodies and 9.80×10⁴ TCID₅₀/mL against NP specific monoclonal antibody.

FIG. 5. Sensitivity of polyPLA. Comparison of polyΔCt and monoΔCt titers with viral culture titers using the nasal swabs collected from the ferrets infected with A/swine/Guangdong/K6/2010(H6N6). After only the first day of infection, a polyΔCt value of 3.20 (±0.06, standard deviation) was obtained, corresponding to a TCID₅₀ titer of 1.00×10³ for the infected ferret. After two days of infection, a polyΔCt value of 5.19 (±0.06) was obtained, corresponding to a TCID₅₀ titer of 1.00×10⁴.

FIGS. 6A-6B. Detecting antigenic variants of H3N2 historical seasonal influenza viruses. JO/33, NA/933, SY/05, BR/10, PE/16, VI/361 representing antigenic cluster BE92, WU95, SY97, BRO7, PE09, and VI11. The homologous polyPLA titers were significantly higher than the heterologous titers for both the antigenic drift event BE92→WU95→WY97 (FIG. 6A) and BR07→PE09→VI11 (FIG. 6B). These results were consistent with those measured from conventional HI and neutralization assays (see TABLE 3 and TABLE 4).

FIGS. 7A-7C. Detecting antigenic variants in human clinical specimens. (FIG. 7A) The percentile of antigenic variants (white portion) compared to their corresponding homologous titers, and a 3-polyPLA-unit threshold was used; (FIG. 7B) the correlation between polyPLA units and HI titers, which were measured using PE/16 polyclonal antisera; (FIG. 7C) phylogenetic tree of HA protein sequences of H3N2 seasonal influenza viruses. The 21 isolates recovered from the 2012-2013 influenza season from Mississippi are marked in red, and the antigenic variants proposed by polyPLA assays using clinical samples against PE/16 are marked with stars. The phylogenetic tree was constructed by maximum parsimony based on HA protein sequences, and genetic clusters were defined based on the reports from Community Network of Reference Laboratories (CNRL) for Human Influenza in Europe (Influenza Virus Characterisation, Summary Europe, June 2012).

FIG. 8 is a simplified flow chart showing the implementation of monoclonal antibody and polyclonal antibodies in an exemplary polyPLA.

FIGS. 9A-9D. Comparison of antigenic characterization of H3N2 swine IAVs using hemagglutination inhibition (HI) assays and polyclonal sera-based proximity ligation assay (polyPLA). (FIG. 9A) Antigenic map derived from HI data. (FIG. 9B) Antigenic map derived from polyPLA data. (FIG. 9C) Correlation of HI titers and polyPLA values; polyPLA values can be predicted from HI titers by the following formula: polyPLA values=1.2665×log₂(HI titers)−0.5569, R²=0.8169. (FIG. 9D) Correlation of the fold changes in HI titers and those in polyPLA values; fold change in polyPLA values can be predicted from fold change in HI titers by the following formula: ΔpolyPLA values=0.9929×Δlog₂(HI titers)−0.0491, R²=0.8494. A total of 7 representative H3N2 swine influenza A viruses (SIVs) were selected to represent antigenic clusters H3SIV-α and H3SIV-β (Table 1). The homologous ferret antisera for these viruses were used to perform the HI assay and polyPLA. The HI assays were performed using 0.5% red blood cells. Antigenic maps were constructed using AntigenMap (http://sysbio.cvm.msstate.edu/AntigenMap). Viral isolates are 09SW64, A/swine/Ohio/095W64/2009 (H3SIV-α); 09SW96, A/swine/Ohio/095W96/2009 (H3 SIV-α); 10SW130, A/swine/Ohio/10SW130/2010 (H3SIV-β); 10SW156, A/swine/Ohio/10SW156/2010 (H3SIV-β); 10SW215, A/swine/Ohio/10SW215/2010 (H3SIV-β); 11 SW208, A/swine/Ohio/11SW208/2011 (H3SIV-β); 11SW347, A/swine/Ohio/11SW347/2011 (H3SIV-β).

FIGS. 10A-10B. Comparison of sensitivity of cell culture based viral titration and polyclonal sera-based proximity ligation assay (polyPLA) in detecting influenza A viruses (IAVs) in nasal wash and nasal swab samples collected from feral swine infected with A/swine/Texas/A01104013/2012(H3N2). (FIG. 10A) Variations of TCID₅₀ and polyPLA titers in 12 swine. Horizontal dashed line indicates 1000 TCID₅₀/mL. (FIG. 10B) Average number of days after virus challenge that virus could be detected by TCID₅₀ and polyPLA. The whiskers of the box-and-whisker plots denote the smallest value to the larger value, while the box extends to the 25^{th} and 75^{th} percentiles, with the median in the middle. The infecting virus belongs to swine influenza A virus (SIV) antigenic cluster H3SIV-β. Swine 2, 3, 4, 6, 7, 8, 11, and 12 were inoculated nasally with virus; swine 1, 5, 9 and 10 were sentinel swine housed in the same room.

FIGS. 11A-11B. Optimization of the polyclonal sera-based proximity ligation assay (polyPLA) in detecting antigenic variants in clinical samples from swine infected with IAV. (FIG. 11A) Distribution of IAV-positive samples (white bars, N=61) vs. IAV-negative samples (grey bars, N=20) obtained using NP monoclonal antibody and various ΔC_{T} values. (FIG. 11B) Distribution of H3SIV-α vs. H3SIV-β samples at various ΔpolyPLA values. 09SW, A/swine/Ohio/09 SW96/2009(H3N2); 10SW, A/swine/Ohio/10 SW215/2010(H3N2); 11SW, A/swine/Ohio/11SW347/2011(H3N2); shown in dark grey, light grey, and white bars, respectively. Data analyses were performed using SAS 9.4 (SAS Institute Inc., Cary, N.C., USA) with 95% CIs.

### DETAILED DESCRIPTION

The following detailed description is presented to enable any person skilled in the art to make and use the invention. For purposes of explanation, specific details are set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

The presently-disclosed subject matter includes a unique antigenic characterization method using polyclonal antibody-based proximity ligation assays (polyPLA). Methods, kits, and other tools disclosed herein are useful in detecting microbial antigenic variants in samples, including clinical samples.

The present invention involves the early detection of antigenic variants for viral, bacterial, parasitic protozoan, and other human or non-human pathogens. Antigenic changes cause ineffectiveness of vaccines and, as a result, vaccines must be updated frequently. The present invention provides methodologies of earlier detection of antigenic variants that allows important decision-making to occur regarding shift of vaccine strains and selection of correct vaccine strains. Thus, it should be appreciated that the methodologies include vaccine design using the results of antigenic variant detection the disclosed methods and kits. Vaccine design and production for a variety of pathogens is well-known in the field. Specifically, the present invention provides novel methods, processes, and kits for detecting microbial pathogen antigenic drift and better protection of both animals and humans. The detection of the antigenic drift(s) of the present invention is the first use of proximity ligation assays (PLAs) to detect antigenic variants using affinity purified polyclonal antiserum. The affinity purified polyclonal antiserum makes the assays more assessable and broadly applicable. More importantly, the invention utilizes polyclonal antibody (antiserum) that makes it possible to characterize antigenic drift and to identify antigenic variants. Previous methods have applied monoclonal antisera to detect the antigens and monoclonal antisera are not effective in detecting antigenic variant characterization. Current methods do not detect antigenic variations using clinical samples directly, as does the present invention.

Identification of antigenic variants is the key to a successful vaccination programs. The empirical serological methods to determine viral (e.g., influenza) antigenic properties require viral propagation. In certain embodiments, the presently-disclosed subject matter includes a unique quantitative PCR-based antigenic characterization method using polyclonal antibody and proximity ligation assays, or so-called polyPLA, was developed and validated. This method can detect a viral titer that is less than 1,000 TCID₅₀/mL. Not only can this method differentiate between different HA subtypes of influenza viruses but also effectively identify antigenic drift events within the same HA subtype of influenza viruses. Applications in H3N2 seasonal influenza data showed that the results from this novel method are consistent with those from the conventional serological assays. This method is not limited to the detection of antigenic variants in influenza but also other pathogens. In some embodiments, the pathogen may be a bacterium, such as Escherichia coli, Campylobacter jejuni, Bordetella pertussis, Haemophilus influenzae, Streptococcus pneumoniae, Neisseria gonorrhoeae, and Neisseria meningitidis, a virus, such as influenza A and HIV, dengue virus, a parasitic protozoan, such as trypanosomiasis and malaria, or others. The foregoing are examples only, and in no way limiting of the utility of the present inventions disclosed herein. It has the potential to be applied through a large scale platform in disease surveillance requiring minimal biosafety and directly using clinical samples. The methodology and process of the present invention is the first to utilize clinical samples without virus isolation, which could provide significant benefits in monitoring vaccine efficacy and identifying emerging antigenic variants. The invention provides diagnosis kits that target various infectious diseases including, but not limited to, influenza diagnosis kits targeting human influenza surveillance and vaccination strain selection, swine influenza surveillance, and vaccine strain selection, avian influenza surveillance and vaccine strain selection, and canine influenza surveillance and vaccine strain selection. The methodologies of the present invention can be used in laboratory diagnosis, disease surveillance, and assessment of vaccine effectiveness for both animal and human subjects in public health and animal health indications.

The present invention provides a novel methodology for detecting viral and bacterial antigenic drift. It is a sensitive, robust, and simple methodology and process that can be applied in disease surveillance, in monitoring vaccine effectiveness, and in identifying emerging antigenic variants. The invention provides for development of a polyclonal antibody-based proximity ligation assay that is used to quantify an antigen-antibody interaction. The novel methodology comprises a proximity ligation assay (PLA), which utilizes quantitative polymerase chain reaction (qPCR) as the quantifying platform, and can, therefore, be applied in most laboratories in the United States and other developed countries. The novel technique can be applied toward influenza viruses and other infectious agents. Particularly helpful, will be the application of the inventive methodologies to those RNA viruses with rapid antigenic changes or shifts. For many pathogens, especially RNA viruses, mutations of surface glycoproteins will lead to antigenic drift and eventually evade the host immune protection. For example, the mutations in influenza surface glycoproteins hemagglutinin and neuraminidase cause antigenic drift since these two genes are the primary targets for host immune systems and these mutations can result in variation of viral antigenicity. These influenza antigenic drift variants allow the virus to escape the host immunity and cause disease outbreaks and epidemics. Rapid detection of novel influenza viruses and sensitive quantification of influenza-antibody interaction would be useful for influenza diagnosis as well as vaccine strain selection. In some embodiments, the PLA assay methods includes: labeling antibodies of interest with biotin (a linker molecule); preparing proximity probes for each antibody; incubating the samples with the proximity probes for about one hour; initiating a ligation reaction; and utilizing a quantitative PCR platform to determine the assay results.

The presently-disclosed subject matter includes a method for detecting antigenic variants of a pathogen, which involves incubating a labeled polyclonal antiserum with a sample containing the pathogen, and quantifying antiserum-pathogen (antibody-antigen) binding avidity. In some embodiments, the IgG are purified from sera using Pierce Chromatography Cartridges Protein AJG.

In some embodiments of the method, the polyclonal antiserum is labeled with at least a pair of oligonucleotide assay proximity probes, such as sodium azide-linked oligonucleotides, which include a pair of primers (3′ and 5′) and a connector oligonucleotide. In some embodiments, the pair of primers are about 35-45 nucleotides long and the connector is about 20-25 nucleotides long.

In some embodiments, there is proximity ligation of the two oligonucleotides following the incubation.

In some embodiments, the antiserum-pathogen (antibody-antigen) binding avidity is quantified using qPCR.

The presently-disclosed subject matter further includes a nucleic acid molecule comprising a pair of primers and a connector. In some embodiments, the nucleic acid molecule also includes a detectable marker. In some embodiments, the nucleic acid molecule includes sodium azide-linked oligonucleotides comprising a pair of primers and a connector. In some embodiments, the pair of primers are about 35-45 nucleotides long and the connector is about 20-25 nucleotides long.

The presently-disclosed subject matter further includes a multiplex assay, which uses a monoclonal antibody targeting a conserved region of a protein family, and involves quantifying the total protein concentrations and then using polyclonal antibodies targeting different testing potential epitopes which can be changed. For example, for influenza A virus, a monoclonal antibody targeting HA2 is used to quantify the total protein amount, the others targeting different influenza subtypes (e.g., H1 and H3) or different clades of H5 viruses (e.g., clade 2, clade 7, etc.), and that the antigenic changes can be tested.

The presently-disclosed subject matter further includes a kit for use in detecting antigenic variants of a pathogen of interest. The kit can include, in some embodiments, a nucleic acid molecule as disclosed herein and reagents for practicing the methods disclosed herein.

As will become apparent to those of ordinary skill in the art upon study of this document, the present inventors have developed a unique high throughput method to detect antigenic variants. Embodiments of this method can detect, for example, an influenza viral titer that is less than 1,000 TCID₅₀/mL. Embodiments of this method have been validated by detecting antigenic drift events in H3N2 viruses. Embodiments of this method allow antigenic characterization using clinical samples. Embodiments of this method can be applied to various viral and other pathogens.

Currently available methods, including HI, MN, and ELISA, have been demonstrated with increased limitations. For example, HI is widely used by the World Health Organizations and influenza vaccine companies to identify influenza antigenic variants but has limitations because viruses are losing the binding ability to certain types of red blood cells. Moreover, MN assay is too time-consuming and not robust. ELISA is not effective in differentiating antigenic characters. Additionally, none of the current methods have been developed to or have the ability to detect antigenic drifts using clinical samples directly. A robust, sensitive, and economic methodology and process is needed for detecting antigenic drift events in both research and clinical platforms. The present invention provides such a methodology, process, and kit for detecting drifts of viral and bacterial pathogens. The invention does not have the limitations of the HI assay or virus strains which are losing the ability to bind RBCs. The invention is more robust and does not require as much time in sample preparation and assay operation. It can detect antigenic drift using clinical samples directly without virus isolation, which is required for the HI assay and the MN assay. The present invention methodology and process can provide commercial kits to test clinical samples directly.

### (1) Hemagglutination Inhibition (HI) Assay.

Mechanism: The antigen in HI tests is simply a solution of the antigenic particles (usually a virus) which is capable of inducing the reaction of hemagglutination when mixed with a suspension of red blood cells. This agglutination is not an antigen/antibody reaction, but rather is the attachment of viral particles by their receptor sites to more than one (1) cell. As more and more cells become attached in this manner, agglutination becomes visible. The presence and concentration of antibody is measured by its ability to inhibit the agglutination at various dilutions.

Reagents: The antigen is usually prepared by growing a naturally hemagglutinating virus (NDV and Avian Influenza viruses) in chick embryo and collecting allantoic fluid (Beard et al., 1975). Occasionally it may be possible to use re-constituted vaccine as a hemagglutinating antigen. If virological examinations are also carried out in the same laboratory or if the viruses used in antigen production are not vaccinal, then it would be important to inactivate the antigen by chemical treatment (though this will reduce the titer of the antigen). Some viruses (e.g., Infectious Bronchitis) though not naturally hemagglutinating can be made to hemagglutinate by treatment with an enzyme treatment. Preparation of such antigens is more complicated since it will normally be required to concentrate virus, usually by ultra-centrifugation. The only other reagent required for carrying out this test is a suspension of red blood cells. Most HI tests carried out in routine poultry serology use chicken erythrocytes. It is usually recommended that the source of the erythrocytes be un-vaccinated chickens. In the UK, a license is required under the Animals (Scientific Procedures) Act, 1986 to collect blood as source of erythrocytes. It is this author's experience that age and vaccination history of the donor has no effect on the results (McMullin, 1979). It is important to carry out at least three (3) wash cycles to ensure that the final suspension is free of antibody and other serum proteins. A fourth cycle is advisable if the HI titers in the source birds are 1:128 or higher. After washing, the red cells are re-suspended in PBS at a standard concentration. The packed red cells should be re-suspended at least at 0.75%, since lower erythrocyte concentrations tend to be associated with more variable HA values for the antigen and hence affect HI results (McMullin, 1979).

Methods: It is possible to carry out rapid hemagglutination tests and hemagglutination inhibition tests on a plate, just as for the bacterial agglutination tests described above. However, HA and HI are generally only used in this way to confirm the presence and identity of a hemagglutinating antigen. Identification and quantification of HI antibody, on the other hand, is nearly always carried out by the equivalent of the slow agglutination test, originally in tubes, now almost always in micro-titer plates. Detailed descriptions of these methods may be found in the literature (Allen and Gough, 1974).

Assays: The commonly-used HI tests in chickens are for Newcastle disease (Paramyxovirus-1), Infectious bronchitis (Coronavirus), and EDS-76 (adenovirus). HI tests may also be carried out for Avian Influenza. However, since there is poor cross-reactivity between the different hemagglutinin groups, AGP is favored for routine screening. Various serotypes of IBV have been used in HI tests as an aid in suggesting the likely infecting strain. This use is complicated by a high degree of cross-reactivity.\par Comments: HI tests require inexpensive reagents though they are labor-intensive. The fact that a series of dilutions are separately tested means that the results are highly reproducible.

### (2) The Microneutralization (MN) Assay.

The microneutralization (MN) assay is another immunological technique used by the Centers for Disease Control and Prevention to determine that some adults have serum cross-reactive antibodies to influenza virus, e.g. the 2009 H1N1 influenza A virus. Viral replication is often studied in the laboratory by infecting cells that are grown in plastic dishes or flasks, commonly called cell cultures. Many viruses kill such cells. As the virus replicates, infected cells round up and detach from the cell culture plate. These visible changes are called cytopathic effects.

There is another way to visualize viral cell killing without using a microscope: by staining the cells with a dye. In the example shown below, cells have been plated in the small wells of a 96 well plate. One well was infected with virus, the other was not. After a period of incubation, the cells were stained with the dye crystal violet, which stains only living cells. It is obvious which cells were infected with virus and which were not.

This visual assay can be used to determine whether a serum sample contains antibodies that block virus infection. A serum sample is mixed with virus before infecting the cells. If the serum contains antibodies that block viral infection, then the cells will survive, as determined by staining with crystal violet. If no antiviral antibodies are present in the serum, the cells will die. In its present form, this assay tells an investigator only whether or not there are antiviral antibodies in a serum sample. To make the assay quantitative, two-fold dilutions of the serum are prepared, and each is mixed with virus and used to infect cells. At the lower dilutions, antibodies will block infection, but at higher dilutions, there will be too few antibodies to have an effect. The simple process of dilution provides a way to compare the virus-neutralizing abilities of different sera. The neutralization titer is expressed as the reciprocal of the highest dilution at which virus infection is blocked.

In the example shown here, the serum blocks virus infection at the 1:2 and 1:4 dilutions, but less at 1:8 and not at all at 1:16. Each serum dilution was tested in triplicate, which allows for more accuracy. In this sample, the neutralization titer would be 4, the reciprocal of the last dilution at which infection was completely blocked.

Microneutralization simply means that the neutralization assay is done in a small format, such as a 96 well plate, instead of larger cell culture dishes.

### (3) Reverse-Transcription PCR.

Another method for identifying antigenic drift is the direct molecular identification of influenza isolates which is a rapid and powerful technique. The reverse-transcription PCR (RT-PCR) allows template viral RNA to be reverse transcribed producing complementary DNA (cDNA) which can then be amplified and detected. This method can be used directly on clinical samples and the rapid nature of the results can facilitate investigation of respiratory illness outbreaks. Genetic analysis of influenza virus genes (especially the HA and NA genes) can be used to identify an unknown influenza virus when the antigenic characteristics cannot be defined.

Genetic analyses can also be used to monitor the evolution of influenza viruses and to determine the degree of relatedness between viruses from different geographical areas and those collected at different times of the year.

### (4) PLA-Based Antigenic Variation Detection Methods.

Demanding a new technique. Influenza virus cause seasonal and pandemic outbreaks and continue to present a threat to public health. Seasonal influenza leads up to, 49,000 deaths and more than 200,000 hospitalizations in the U.S. each year, and an influenza pandemic may cause loss of from thousands to millions of human lives in a short time period. The mutations in influenza surface glycoproteins hemagglutinin (HA) and neuraminidase (NA) cause antigenic drift because these two genes, especially HA, are the primary targets for host immune systems, and these mutations can result in variation of viral antigenicity. On the other hand, antigenic shift changes the antigenic properties by replacing an HA or NA genes from other influenza A viruses, usually animal origin influenza A viruses, and the antigenic shift can lead to influenza pandemics.

Vaccine is the primary option to counteract and reduce the impacts of influenza outbreaks. Since influenza viruses are continuously changing their antigenicity in order to escape the host immunity, the vaccine strains almost need to be updated annually to obtain antigenic matches between the vaccine strain and the strain potentially causing future outbreaks. Detecting of influenza antigenic variants are keys to a successful influenza vaccination program for both pandemic preparedness and seasonal influenza prevention and control.

Immunological tests, such as hemagglutinin inhibition (HI) assay and microneutralization (MN) assay, are usually utilized to identify antigenic variants among the circulating strains. Among these assays, MN seems to be more sensitive and specific than HI, but it is time consuming and sometimes difficult to perform under biosafety-lever 3 (BSL-3). For this reason, MN is generally used to validate the results from HI. Because of its simplicity, HI has been one of the routine procedures in influenza vaccine selection. HI assay is an experiment to measure how a test influenza antigen and a reference antigen (e.g., a current vaccine strain) match through the immunological reaction between the test antigen and the reference antiserum, which is usually generated in ferret using the reference antigen. HI experiment has a limitation because it uses red blood cells (RBCs), e.g., turkey red blood cells, as indicators for binding affinity of antigen and antiserum: a higher interaction between antigen and antisera will lead to a less hemagglutination of RBCs.

However, HI data is less robust. For example, RBCs used from different species and even variation in RBC lots can give varied results; the data are subjective interpretations; and the HI assays have difficulties automating and standardizing operation. More importantly, mutations of the receptor binding site in HA (antigenic drift) are causing H1N1 and H3N2 to lose the ability to bind to certain types of RBCs. For example, isolates for 1992 H1N1 lost the ability to agglutinate chicken RBCs due to four amino acid changes that had already occurred in 1988. Minority antigenic variants within a heterogeneous population cannot be assessed by the serological method of HI. In addition, emerged mutations due to adamantanes resistance can affect the binding affinity of red blood cells thus the HI titers. Thus, there is a demand of red blood cell independent assay for influenza antigenic variation.

Preparation of polyclonal antisera: The polyclonal antisera were generated using 4- to 6-month-old ferrets (Triple F Farm, Pa.), but they can be generated by inoculating any appropriate host with a sample pathogen. The ferrets were anesthetized with isoflurane and inoculated intranasally with 106 50% egg infectious doses (EID5Os) of a H3N2 challenge virus. The ferret sera were collected after three weeks post infection. The IgG of the ferret sera against human H3N2 were purified by Pierce Chromatography Cartridges Protein A/G according to the manufacturer. Besides ferrets, the polyclonal antisera could be also collected from human subjects or generated in other animals (for example, pigs).

### Steps of PLA Assay

A. Antibody labeled with biotin: The purified ferret sera and monoclonal antibodies were biotinylated with Biotin-XX Microscale Protein Labeling Kit according to the manufacturer.

B. Proximity probe preparation: Two assay probes for each antibody were prepared by combining the biotinylated antibodies with either 3′ or 5′ TaqMan Prox-Oligo (probe A and probe B). For example, for each probe, 2.5 μL 200 nM biotinylated antibody was combined with 2.5 μL 200 nM 3′ Prox-Oligo together, and the mixture incubated at room temperature for 60 minutes. Then 45 μL, Assay Probe Storage Buffer was added, briefly centrifuged, and incubated at room temperature for 20 minutes.

C. Incubate sample with proximity probes: The proximity ligation assays (PLA) were performed by first diluting equal parts of probes A and B mixture 1:10 with phosphate-buffered saline (PBS, pH 7.4), which 2 μL were then combined with 2 μL, diluted lyzed virus. The mixture was centrifuged briefly and incubated at 37° C. for one hour.

D. Ligation reaction: Then 96 μL ligation solution was added to the probe and virus mixture and incubated again at 37° C. for 10 minutes, cooking at 4° C. for 10 minutes.

E. Quantitative PCR: After incubation, 9 μL were transferred to a new 0.2 mL microcentrifuge tube with 11 μL, Real-Time PCR mix (10 μL Fast Master Mix, 2× plus 1 μL Universal PCR Assay, 20×) and was briefly centrifuged. The quantitative polymerase chain reaction (PCR) cycling was as follows: 95° C. for 2 minutes, 40 cycles of 95° C. for 15 seconds, 60° C. for 1 minute; quantitative PCR was carried on a Stratagene QPCR machine.

### Normalization of Viral Quantity Using NP Genes

The viruses were lyzed before performing PLA assay to quantify NP proteins. The NP proteins were detected using monoclonal antibody.

### Detection of Influenza Antigenic Drifts

To make sure (1) the influenza viral quantity is equal; (2) the viral concentration is not saturated, the viruses are diluted to have a PLA ΔCt-NP value of 4.0-5.0. Then the viruses are used to react with polyclonal antisera. The resulting ΔCt reflect the affinity of antibody-antigen interactions. A higher ΔCt reflects a higher binding affinity between influenza virus and the corresponding antibody. In most cases, the samples can be subjected directly without dilution to PLA with polyclonal antisera, but the resulting ΔCt will be normalized by PLA ΔCt-NP to ensure the viral quantity across the testing samples is equal.

This assay can be used to target a number of antigens and antisera, and the resulting ΔCt table can reflect the antigenic relationship between the testing antigens. This table is similar to HI table and MN table.

While the terms used herein are believed to be well understood by those of ordinary skill in the art, certain definitions are set forth to facilitate explanation of the presently-disclosed subject matter. Unless defined otherwise, all technical and scientific terms used herein have the same meaning as is commonly understood by one of skill in the art to which the invention(s) belong. As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

The presently-disclosed subject matter is further illustrated by the following specific but non-limiting examples. The following examples may include compilations of data that are representative of data gathered at various times during the course of development and experimentation related to the present invention.

### EXAMPLES

### Materials and Methods

Viruses and antibodies. The H3N2 viruses used in this study were obtained from the Centers of Disease Control and Prevention, Department of Health & Human Services and BEI Research Resources Repository (http://www.beiresources.org/) (TABLE 1), and the monoclonal antibodies against nucleoprotein (NP) from Millipore, United States. The viruses were propagated at Madin-Darby Canine Kidney (MDCK) cells and stored at −80° C. before usage. The polyclonal antisera were generated using 4- to 6-month-old ferrets (Triple F Farm, PA). The ferrets were anesthetized with isoflurane and inoculated intranasally with 10⁶ 50% egg infectious doses (EID₅₀) of a challenging virus. The ferret sera were collected three weeks post-infection. The viral isolation was performed using MDCK cells.

TABLE 1
The H3N2 influenza A viruses used in the Examples.

| Virus | Abbreviation | Antigenic Cluster^{a} |
|---|---|---|
| A/Sichuan/2/87(H3N2) | SI/2 | ND |
| A/Johannesburg/33/94(H3N2) | JO/33 | BE92 |
| A/Nanchang/933/95(H3N2) | NA/933 | WU95 |
| A/Sydney/05/97(H3N2) | SY/05 | SY97 |
| A/Brisbane/10/07(H3N2) | BR/10 | BR07 |
| A/Perth/16/09(H3N2) | PE/16 | PE09 |
| A/Victoria/361/11(H3N2) | VI/361 | VI 11 |

Note:
^{a}antigenic cluster was described in Sun et al. (2013); and ND, not determined.

Labeling of antibodies. IgG were purified from ferret polyclonal antisera and mice monoclonal antibodies using Pierce Chromatography Cartridges Protein A/G according to the manufacturer's instruction (Pierce, Rockford, Ill.) and then biotinylated with Biotin-XX Microscale Protein Labeling Kit according to the manufacturer's directions (Life Technologies, Carlsbad, Calif.). To remove the free biotin, Slide-A-Lyzer Dialysis Cassettes (Thermo Scientific Pierce, Rockford, Ill.) were used; dialysis was performed at 4° C. in cold 1×PBS (pH 7.4), and the buffer was changed at least five times each 2 hours.

Forced proximity probe test. An aliquot of biotinylated antibody stock solution was diluted to 200 nM (30 μg/mL); 2 μL of diluted biotinylated antibody was added to 2 μL of equal mixture of 200 nM 3′ and 5′ TaqMan Prox-Oligo, designated probe A and probe B (Life Technologies, Carlsbad, Calif.), and incubated at room temperature for 60 minutes. A negative control was made replacing diluted biotinylated antibody with 2 μL antibody dilution buffer. After incubation, 396 μL of assay probe dilution buffer was added and incubated for another 30 minutes at room temperature. Then 96 μL of ligation solution was added to 4 μL of the probe and virus mixture, incubated again at 37° C. for 10 minutes, and cooled at 4° C. for 10 minutes. After incubation, 9 μL were transferred to a new 0.2 mL microcentrifuge tube with 11 μL qPCR mix (10 μL Fast Master Mix, 2× plus 1 μL Universal PCR Assay, 20×) and was briefly centrifuged. The qPCR cycling was as follows: 95° C. for 2 minutes, 40 cycles of 95° C. for 15 seconds, and 60° C. for 1 minute.

The change in threshold cycle (ΔCt) values was calculated for each biotinylated antibody: Average Ct (negative control)−Average Ct (forced proximity probe). If the ΔCt≧8.5, the test biotinylated antibody was considered suitable for use in the PLA.

Probe preparation. Two assay probes for each antibody were prepared by combining the biotinylated antibodies with either 3′ or 5′ TaqMan Prox-Oligo (probe A and probe B). For example, for each probe, 2.5 μL 200 nM biotinylated antibody was combined with 2.5 μL of either 200 nM 3′ Prox-Oligo or 200 nM 5′ Prox-Oligo, and the mixture was incubated at room temperature for 60 minutes. Then, 45 μL Assay Probe Storage Buffer was added, briefly centrifuged, and incubated at room temperature for 20 minutes.

PLA and quantification of polyΔCt and monoΔCt. The TaqMan PLA was performed by first diluting equal parts of probes A and B mixture 1:10 with phosphate-buffered saline (PBS, pH 7.4). For the non-protein control (NPC), 2 μL were combined with 2 μL diluted virus (virus lyzed for NP detection) or 2 μL 1×PBS, pH 7.4. The mixture was centrifuged briefly and incubated at 37° C. for one hour. Then, 96 μL, of ligation solution was added to 4 μL of the probe and virus mixture, incubated again at 37° C. for 10 minutes, and cooled at 4° C. up to 10 minutes. After incubation, 2 μL of 1× protease was added to each ligation reaction and incubated at 37° C. for 10 minutes, 95° C. for 5 minutes, and 4° C. for holding. Lastly, 9 μL of the product was transferred to a new 0.2 mL microcentrifuge tube with 11 μL qPCR mix (10 μL Fast Master Mix, 2× plus 1 μL, Universal PCR Assay, 20×) and was briefly centrifuged. The qPCR cycling was as follows: 95° C. for 2 minutes, 45 cycles of 95° C. for 15 seconds, and 60 ° C. for 1 minute.

The NPC was used as a reference background and the threshold cycle (Ct) value given dictated the non-target ligation signal noise of the assay. A total of three replicates for each sample and control were performed. To calculate the ΔCt values: Average Ct (NPC)−Average Ct (sample), which represented the true target-mediated signal above background. The cutoff of ΔCt≧3.00 was used for qualitative analysis of viral and antibody binding, as according to the TaqMan Protein Assays Sample Prep and Protocol (2013).

HI and virus neutralization assays. In the HI assay, the receptor destroying enzyme (RDE, Denka Seiken Co., Japan) was used to treat the ferret sera in the ratio of 1:3 (RDE: sera, volume: volume) for 18 hours at 37° C., then heat inactivated at 56° C. for 30 minutes. The treated ferret sera were diluted to 1:10 with phosphate-buffered saline (PBS) then 2-fold serial diluted and reacted with 4-hemmagglutination-units viruses. The HI titers were expressed as the reciprocal of the highest dilution at which virus binding to the 0.5% turkey red blood cells (RBC) was blocked.

For the virus neutralization assay, serially diluted ferret sera were first incubated with 100 TCID₅₀ viruses at 37° C. for 1 hour. The virus-sera mixtures were then adsorbed to MDCK cells for 1 hour. The infected cells were washed twice with PBS buffer and replenished with Opti-Mem Reduced Serum Media (Life Technologies, US). The supernatants from the infected cells were harvested 4 days post-infection and were analyzed using hemagglutination assay.

Data analysis. To compare the antigenic properties across the testing antigens (viruses), the polyPLA unit between antigen (virus) and antibody were computed using the following equation:

polyPLA=a*(polyΔCt−monoΔCt)+b

To improve the computation, a=1.00 and b=10.00 were used. The b=10.00 enabled me to avoid negative numbers. If monoΔCt<3.00, polyPLA will be assigned as “<0”, meaning that the viral loads were too low for analyses. As mentioned above, in general, polyPLA is sensitive in detecting viral loads of approximately 10³ TCID₅₀/mL.

Genomic sequencing and GenBank accession number. The HA genes of 21 H3N2 isolates recovered from human clinical specimens were sequenced using Sanger Sequencing, and they were deposited in GenBank with the accession numbers KM244531-KM244551.

Molecular characterization and phylogenetic analyses. The multiple sequence alignments were conducted using the MUSCLE software package (Edgar, 2004). The phylogenetic analyses were performed using maximum likelihood by GARLI version (Zwickl, 2006), and bootstrap resampling analyses were conducted with 1,000 runs using PAUP*4.0 Beta (Swofford, 1998) with a neighborhood joining method, as previously described (Wan et al., 2008).

### Results

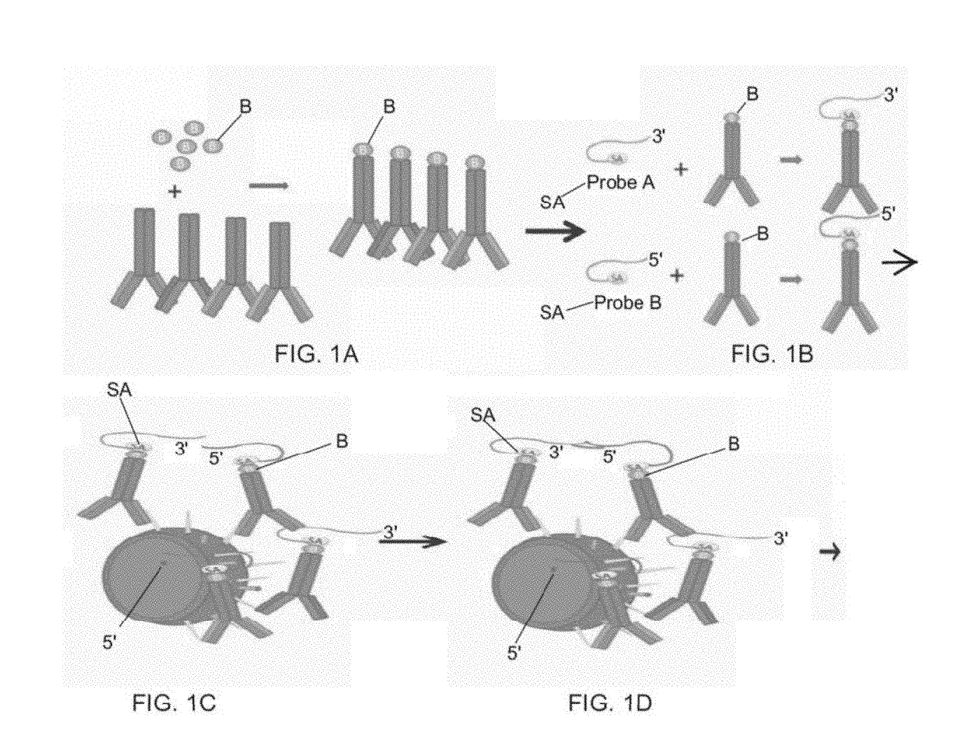

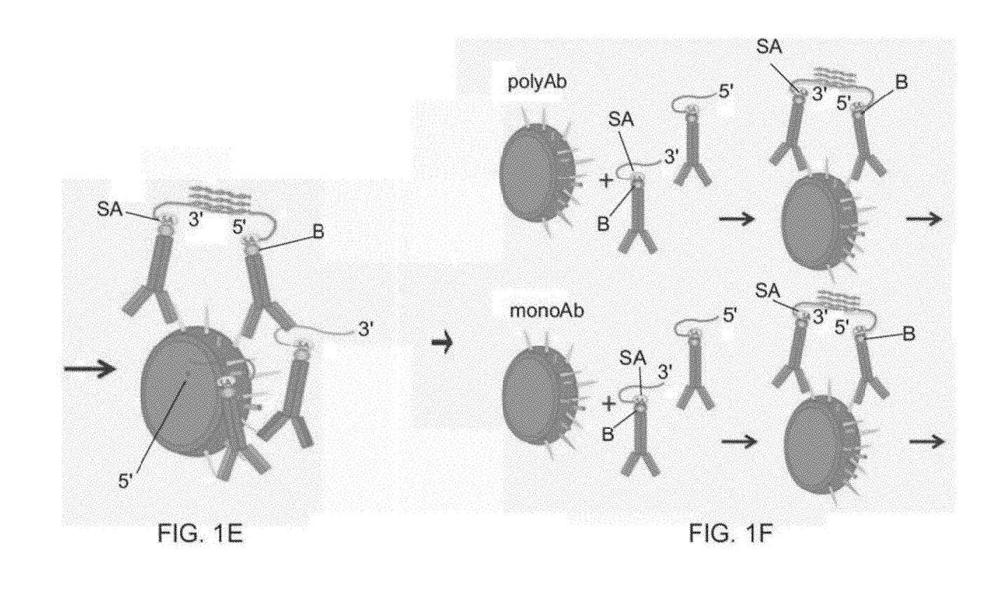

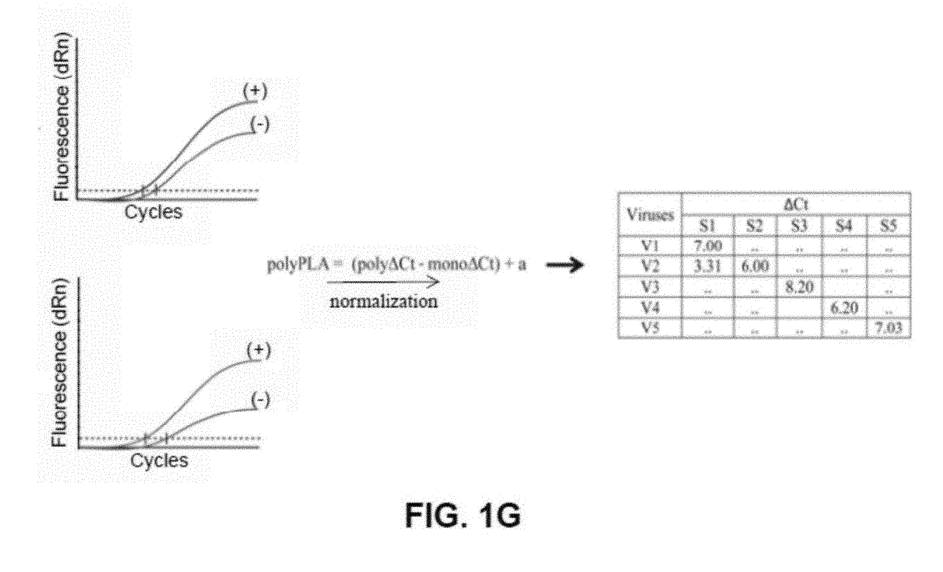

polyPLA for influenza antigenic variant detection. polyPLA quantifies the antibody antigen binding avidity using the amplification signals in quantitative PCR (qPCR) from the pairs of primers attached to a reference polyclonal antiserum. The first step of this experiment is to biotinylate a reference polyclonal antiserum (FIG. 1A), which will then be labeled with sodium azide-linked 5′ and 3′ oligonucleotides (FIG. 1B). The ligation efficiency will be assessed with qPCR. A labeled polyclonal antiserum with ΔCt≧8.5 in the ligation efficiency test is then incubated with a reference antigen (virus) or a testing antigen (FIG. 1C), followed by the proximity ligation of the two oligos (FIG. 1D). The antibody antigen binding avidity is quantified using the amplification signals ΔCt in qPCR (FIG. 1E). The ΔCt values among the polyclonal antisera and antigens can be compared to assess antigenic differences among these tested antigens. These ΔCt values can be viewed as similar to the serological titers, such as HI and neutralization titers, from conventional serological assays (FIG. 1F).

To make the ΔCt values comparable across reference sera, the testing antigens have the same quantities across quantification assays. In HI assays, the antigens are usually standardized to be 4 or 8 units of hemagglutination titer before HI; in neutralization assays, antigen quantities are usually standardized using TCID₅₀(2013). In this assay, the quantities of nucleoproteins (NPs) were used to normalize the amount of viruses in the analyses. For data consistency, a monoclonal antibody targeting conserved regions of NPs was used in the proximity ligation assay (Schlingemann et al., 2010). Thus, for a testing antigen, the polyPLA units were normalized by its ΔCt values for polyclonal antiserum (polyΔCt) with its ΔCt values for monoclonal antibody against NP (monoΔCt) (FIG. 1G).

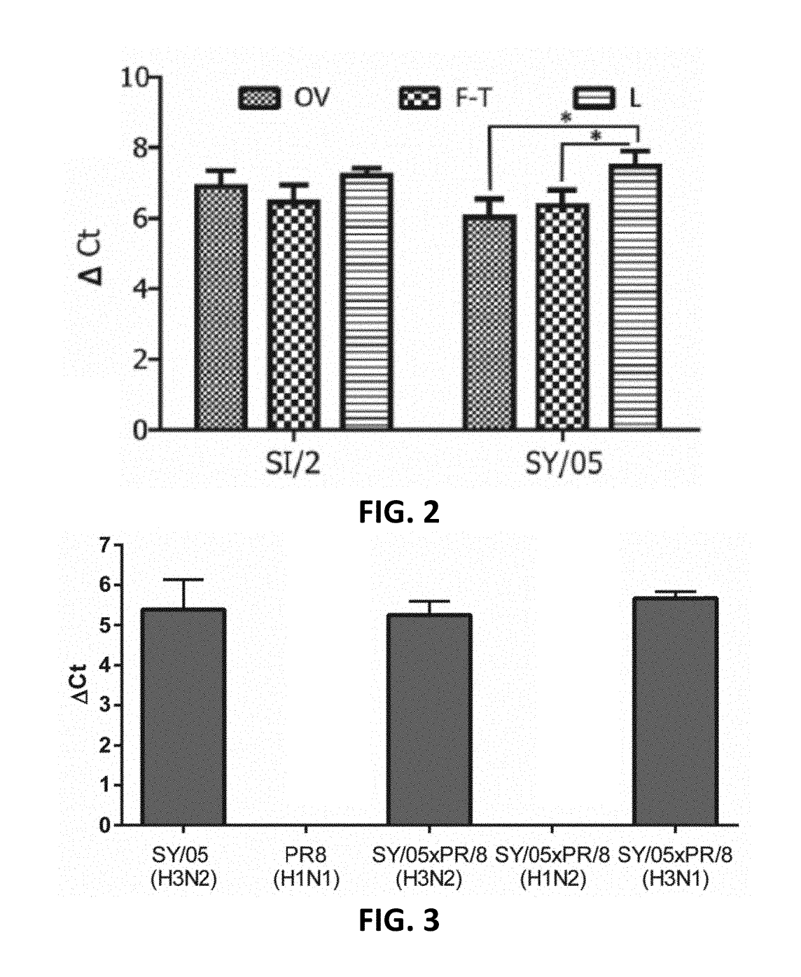

Viral particles must be completely lysed to release NPs and allow for an accurate measure of these protein quantities. Two commonly used methods for viral lysis were compared: freeze/thaw and treatment with lysis buffer. The results showed that lysis buffer treated virus has a significantly higher ΔCt value of 7.46 (±0.45) for A/Sydney/05/1997 (SY/05), p<0.05 (FIG. 2) compared to the freeze/thaw method of viral lysis. However, for A/Sichuan/2/1987 (SI/2), lysis buffer treated virus did not have a significantly different ΔCt value compared to the freeze/thaw method of viral lysis. In the following assays, all the samples used in normalization were treated with lysis buffer.

HA specific IgG predominates polyclonal antisera. polyPLA quantifies the interactions between influenza viral proteins and all IgG present in the polyclonal antisera. To assess the impacts of NA and other internal proteins on polyPLA, three reassortants were constructed between SY/05 and PR8, including SY/05xPR8(H3N2), SY/05xPR8(H3N1), and SY/05xPR8(H1N2). The signals from NPC were used as the control to calculate ΔCt value from proximity ligation assays. The results showed that SY/05, SY/05xPR8(H3N2), and SY/05xPR8(H3N1) had ΔCt values of 5.40(±0.74), 5.67(±0.17), and 5.26(±0.34), respectively (FIG. 3). The ΔCt values from PR8 and the reassortant SY/05xPR8(H1N2) were negligible.

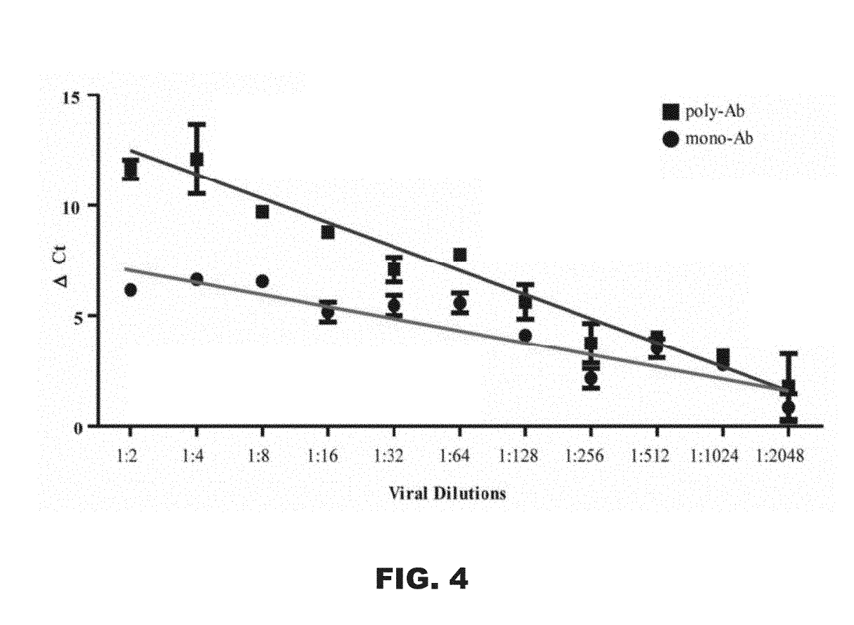

Viral quantities are linearly correlated with ΔCt values. To assess the sensitivity of polyPLA, PLA was performed on influenza A viruses with serial dilutions. Regression analyses demonstrated that the polyΔCt values are linearly correlated with the influenza viral quantities, with Pearson's coefficient R=0.98 for the testing strain SY/05 (p<0.001) (FIG. 4). The cutoff ΔCt value 3.00 was equivalent to 4.90×10⁴ TCID₅₀/mL against its homologous polyclonal antibodies. Similarly, the monoΔCt values were also linearly correlated with HA titers, and the R was 0.92 for SY/05 (p<0.001). The cutoff ΔCt value was equivalent to 9.80×10⁴ TCID₅₀/mL against the NP-specific monoclonal antibody. Similar linear correlations were also observations in A/Johannesburg/33/1994(H3N2) (JO/33) and A/Nanchang/933/1995(H3N2) (NA/933) (data not shown). Linear correlation between viral quantities and ΔCt allows an investigator to normalize the viral titers by using a simple equation such as a*(polyΔCt−monoΔCO+b, where a and b are constant parameters. This normalization method enables an investigator to compare the antigenic properties between the testing antigens (viruses) without justifying the viral quantities before measuring polyΔCt, having been used in HI and neutralization assays to ensure the equivalency of the viral quantities before assays.

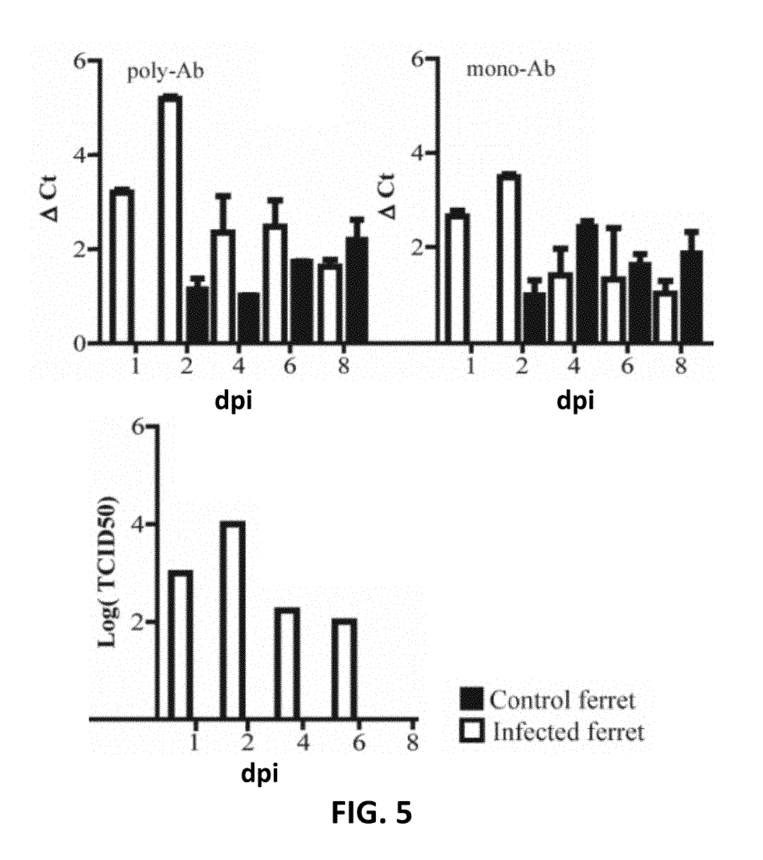

Sensitivities of polyPLA. To test the sensitivity of polyPLA, the viral loads from nasal swabs collected from ferrets infected with A/swine/K6/2011(H6N6) were evaluated. After only the first day of infection, a polyΔCt titer of 3.20 (±0.06, standard deviation) was obtained, corresponding to a TCID₅₀ titer of 1.00×10³for the infected ferret (FIG. 5). After two days of infection, a polyΔCt value of 5.19 (±0.06) was obtained, corresponding to a TCID₅₀ titer of 1.00×10⁴. A higher polyΔCt titer corresponded to a higher TCID₅₀ titer among the nasal wash samples post-infection. All the samples collected from the control ferrets had polyΔCt titers of less than 3 00, and no viruses were recovered from these control ferrets (FIG. 5). Thus, this method is sensitive sufficiently to detect not only the viruses propagated from the laboratory, but also those in animal specimens. The detection limit is approximately 10³ TCID₅₀/mL, which is much less than the viral loads from most patients at the peak time of virus shedding. For example, man can shed 2.6, 5.0, 5.1, 4.9, 3.8, and 1.9 log₁₀ TCID₅₀/mL from one through six days post inoculation of H1N1 seasonal influenza virus, respectively (Baccam et al., 2006).

Detecting antigenic variants of H3N2 historical seasonal influenza viruses. The H3N2 viruses have been causing seasonal epidemic outbreaks since its first introduction into the human population, resulting in the pandemic of 1968. During the past four decades, at least 12 antigenic drift events have been detected; six of which occurred from 1997 to 2010 (Skowronski et al., 2007; Sun et al., 2013). In this study, six historical H3N2 isolates, JO/33, NA/933, SY/05, A/Brisbane/10/2007 (BR/10), A/Perth/16/2009 (PE/16), A/Victoria/361/2011(VI/361) representing antigenic cluster BE92, WU95, SY97, BRO7, PE09, and VI11 (Sun et al., 2013), and their corresponding homologous ferret antisera were used to validate polyPLA. The method was expected to identify significant differences in homologous and heterologous polyPLA titers.

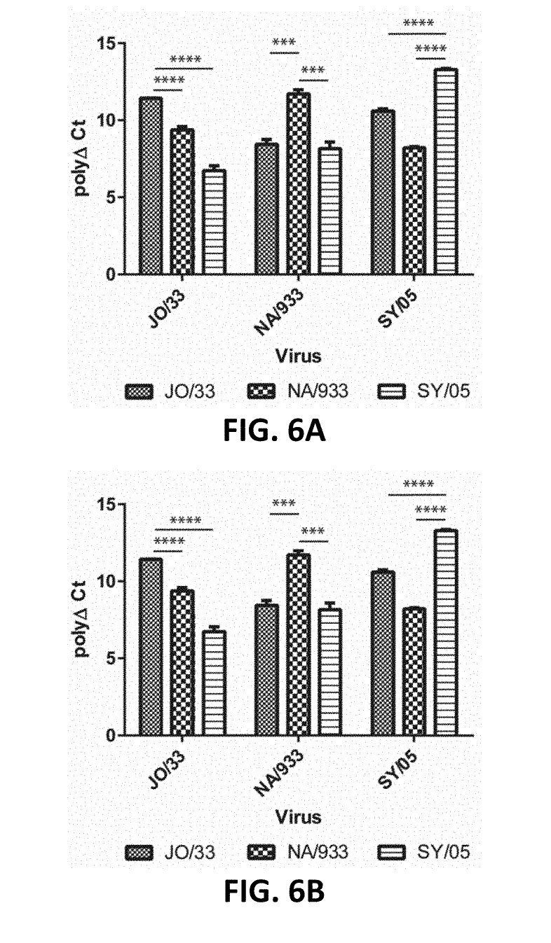

These results showed that the homologous titers were approximately 10.00 polyPLA units. The polyPLA titers for NA/933 and SY/05 against JO/33 antisera were 9.37 (±0.22) and 6.725 (±0.32) polyPLA units, which were significantly less than the homologous JO/33 titer 11.43 (±0.01) units, p<0.0001 (FIG. 6A). The homologous titer for NA/933 was 11.71 (±0.28) whereas the titers for JO/33 and SY/05 against NA/933 antisera were 8.44 (±0.32) and 8.16 (±0.43) units, respectively; the titers for JO/33 against NA/933 antisera were significantly less than the homologous titers for JO/33, p<0.001. The homologous titer for SY/05 was 13.19 (±0.06) units whereas the titers for JO/33 and NA/933 against SY/05 antisera were 10.59 (±0.15) and 8.21 (±0.07) units, respectively; the titers for both JO/33 and NA/933 against SY/05 antisera were significantly less than the homologous titers for SY/05, p<0.0001.

Similarly, the homologous titers for BR/10 were 9.91 (±0.16) whereas the titers for PE/16 and VI/361 against BR/10 sera were 7.97 (±0.12) and 7.72 (±0.21), respectively (FIG. 6B); the titers for PE/16 and VI/361 against BR/10 sera were significantly less than the homologous titers for BR/10, p<0.001. PE/16 had the highest polyΔCt value of 12.00 (±0.20) against PE/16 sera whereas the titers for BR/10 and VI/361 against PE/16 sera were 7.92 (±0.10) and 10.01 (±0.15), respectively; the titers for BR/10 and VI/361 against PE/16 sera were significantly less than the homologous titers for PE/16, p<0.001. The homologous titer for VI/361 was 10.87 (±0.22) whereas the titers for BR/10 and PE/09 against VI/361 sera were 5.90 (±0.10) and 7.93 (±0.15), respectively; the titers for BR/10 and PE/09 against VI/361 sera were significantly less than the homologous titers for VI/361, p<0.001.

Detecting antigenic variants in human clinical specimens. To test the applicability of polyPLA in clinical samples, this same method was applied to characterize antigenic profiles of H3N2 influenza A viruses, which came directly from clinical samples collected in the 2012-2013 influenza season. A total of 100 nasal swabs were collected from September of 2012 to April of 2013, and confirmed as H3N2 positive using quantitative RT-PCR.

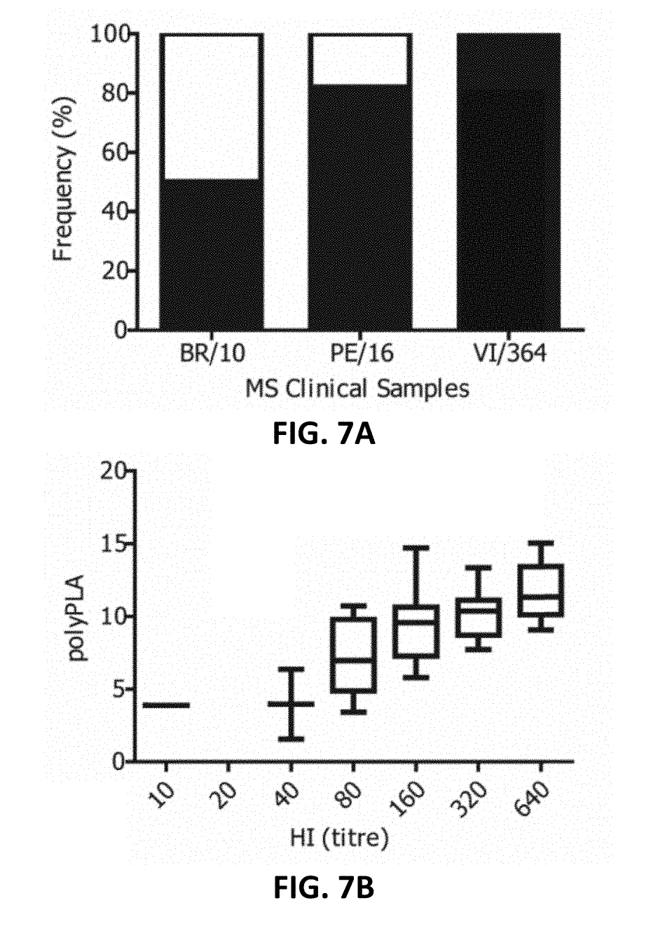

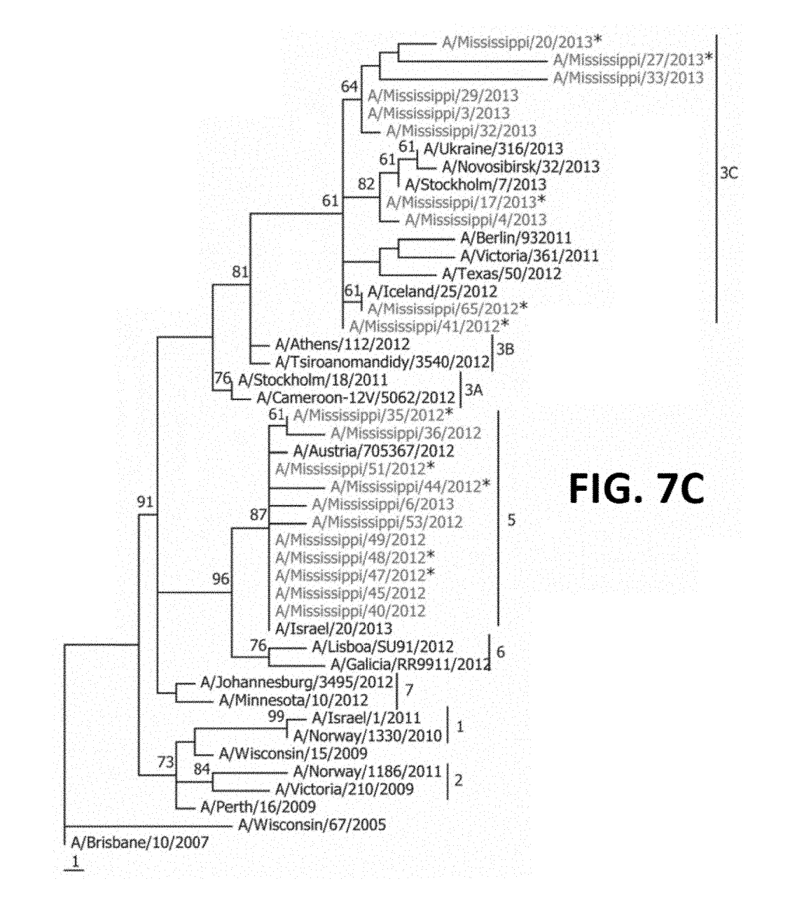

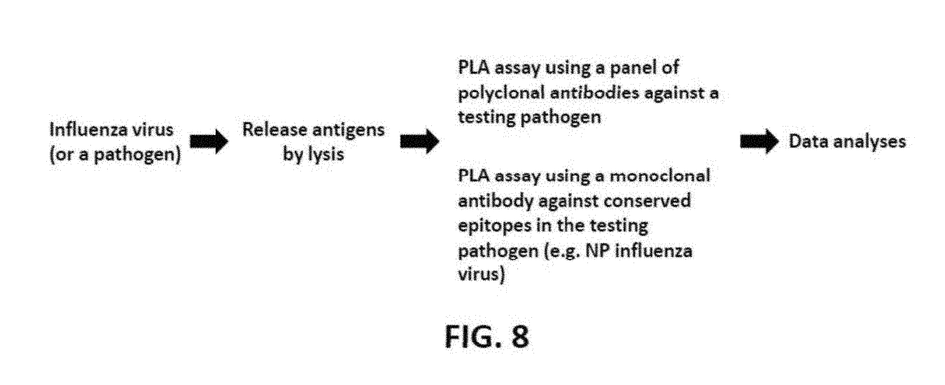

About 50% of these samples had at least a 3-polyPLA-unit decrease compared to BR/10 homologous titers and that about 18% and 1% of these samples had at least 3-polyPLA-unit decrease when compared to PE/16 and VI/361 homologous titers, respectively (FIG. 7A). The polyPLA titers of the majority clinical samples were highest when using VI/361 sera, followed by PE/16 and BR/10. The polyΔCt titers of the clinical samples were compared with the HI titers for these 21 isolates, and the HI titers were positively correlated with polyΔCt titers (FIG. 7B).

To better understand the antigenic and genetic background of the H3N2 viruses in these samples, 21 samples were randomly selected and subjected to viral isolation using MDCK cells. A total of 21 viruses were recovered, and the HA sequences of these viruses were sequenced. The sequence analyses showed that no consistent mutations at the reported antibody binding sites (Wilson and Cox, 1990) were observed in the isolates recovered from this study (TABLE 2).

Since 2007, the H3N2 seasonal influenza viruses have six genetic clusters (2013). The genetic clusters 1 and 2 were PE/16 like-viruses. The viruses from 2010 were scattered into genetic clusters 3, 4, 5, and 6. The phylogenetic analyses showed that these 21 viruses belonged to genetic cluster 3C and 5 (FIG. 7C).

### Discussion

Identification of antigenic variants in disease surveillance is essential, and an ideal antigenic characterization platform should meet the following four criteria: (1) robust, the results should be repeatable; (2) simple and economical, the methods can be carried out in a common diagnosis laboratory; (3) high-throughput, the method should be able to perform on a large-scale; and (4) sensitive, the method will have optimal performance in identifying antigenic variants directly from clinical samples, avoiding viral isolation. For many diseases, the pathogen isolation process is not only time-consuming, but also can change virus antigenic properties, resulting in data that does not accurately represent those antigenic properties in circulating viruses. Furthermore, some pathogens cannot even be recovered from the specimen. The polyPLA developed in this study was designed to meet these four criteria, as confirmed in influenza antigenic variant identification in this study.

The polyPLA quantifies antibody antigen interactions for influenza viral proteins, including both HA and NA, against their corresponding antibodies in the antisera. The principle of this method is more similar to neutralization assays rather than HI assays. More importantly, it can avoid the red blood cell binding problems usually seen in HI assay. For example, egg-adaptation substitutions affect the architecture of the

HA receptor-binding site and alter the interactions of the HA with the terminal sialic acid moiety (Gambaryan et al., 1999).

The high concentration of influenza A viruses could lead to the saturation of viruses over antibody, which is 200 nM used in this study. For example, when the undiluted, MDCK cell derived NA/933 virus was used, there was no significant difference between the ΔCt values against NA/933 antibody and those' against JO/33 antibody (data not shown). After the NA/933 viruses were diluted to 1:40, there was significant difference between the ΔCt values against NA/933 and those against JO/33 antibodies (TABLE 3 and FIG. 6). However, this should not affect the application of this method in clinical samples, from which the virus loads are much smaller than a single isolate.

TABLE 3
The correlation among the titers from polyPLA and those from HI and MN
assays for 10/33, NA/33, and SY/05.

| | Ferret Antisera^{a} | | | | | | | | |
|---|---|---|---|---|---|---|---|---|---|
| | JO/33 | | | NA/933 | | | SY/05 | | |
| | | | polyΔCt | | | polyΔCt | | | polyΔCt |
| | | | (Standard | | | (Standard | | | (Standard |
| Virus | HI | MN | Deviation) | HI | MN | Deviation) | HI | MN | Deviation) |
| JO/33 | 640 | ND | 11.43(0.01) | 40 | ND | 8.44(0.32) | <10 | 40 | 10.59(0.15) |
| NA/933 | 160 | ND | 9.37(0.22) | 1280 | ND | 11.71(0.28) | 160 | 30 | 8.21(0.07) |
| SY/05 | <10 | ND | 6.725(0.32) | <10 | ND | 8.16(0.43) | 1280 | 1280 | 13.29(0.06) |

Note:
^{a}the number in bold is the homologous titer.

TABLE 4
The correlation among the titers from polyPLA and those from HI and MN
assays for BR/10, PE/16, and VI/361.

| | Ferret Antisera^{a} | | | | | | | | |
|---|---|---|---|---|---|---|---|---|---|
| | BR/10 | | | PE/16 | | | VI/361 | | |
| | | | polyΔCt | | | polyΔCt | | | polyΔCt |
| | | | (Standard | | | (Standard | | | (Standard |
| Virus | HI | MN | Deviation) | HI | MN | Deviation) | HI | MN | Deviation) |
| WI/67 | ND | ND | | 40 | 160 | | 320 | 320 | |
| BR/10 | 1280 | 2560 | 9.91(0.16) | 40 | 160 | 7.92(0.10) | 40 | 40 | 5.90(0.10) |
| PE/16 | 80 | 80 | 7.97(0.12) | 640 | 640 | 12.00(0.20) | 160 | 160 | 7.93(0.15) |
| VI/361 | 20 | 20 | 7.72(0.21) | 320 | 640 | 10.01(0.15) | 640 | 640 | 10.87(0.22) |

Note:
^{a}the number in bold is the homologous titer.

Compared to seasonal influenza virus surveillance, the antigenic characterization of emerging pathogens for pandemic preparedness, especially those pathogens in areas without sufficient biosafety facilities, has been challenging. Propagation of these emerging viruses usually requires a high biosafety containment such as BSL-3 and even BSL-4. In most cases, the specimens need to be shipped to a laboratory with appropriate biosafety containment, and sometimes even the paperwork for collaborative agreements, especially among countries, can cause delays when an outbreak occurs. Because the polyPLA can use the clinical samples directly and also use a common qRT-PCR platform, it can perform large-scale analyses with minimal biosafety requirements in laboratory conditions. For example, Biosafety Level 2 level will meet for the polyPLA in influenza surveillance. Thus, this method will be very useful in detecting antigenic variants for the H5N1 highly pathogenic avian influenza viruses and emerging H7N9 low pathogenic avian influenza viruses. In resource limited areas, although the RBCs in conventional serological assays such as HI can be more accessible, it would be much easier and economic to set up a qRT-PCR platform than a high containment environment for viral propagation. Additional supporting data is set forth below in TABLE 5.

TABLE 5
The results of PolyPLA and HI assays for the clinical samples from
Mississippi in the 2012-2013 influenza season.

| | Polyclonal Antiserum | | | | | | | | |
|---|---|---|---|---|---|---|---|---|---|
| | A/Brisbane/10/2007 | | | A/Perth/16/2009 | | | A/Victoria/361/2011 | | |
| sample | HI | polyPLA | SD | HI | polyPLA | SD | HI | polyPLA | SD |
| 3 | 320 | 11.96 | 0.32 | 640 | 13.42 | 1.35 | 320 | 11.14 | 0.18 |
| 4 | 160 | 14.72 | 0.12 | 640 | 13.13 | 0.27 | 320 | 11.0633 | 0.09 |
| 17 | 80 | 4.69503 | 0.09 | 160 | 13.31 | 0.05 | 160 | 9.57 | 0.28 |
| 20 | 10 | 3.87 | 0.83 | 320 | 10.41 | 0.28 | 160 | 9.49 | 0.16 |
| 26 | 80 | 7.65 | 0.33 | 640 | 15.05 | 0.19 | 320 | 9.73 | 0.07 |
| 27 | 320 | 4.3 | 0.97 | 640 | 10.13 | 0.12 | 320 | 8.97 | 0.24 |
| 30 | 40 | 1.55167 | 0.33 | 160 | 6.09 | 0.43 | 80 | 9.45 | 0.16 |
| 32 | 160 | 7.345 | 0.43 | 640 | 9.07 | 0.12 | 320 | 10.39 | 0.25 |
| 33 | 80 | 6.97 | 0.25 | 160 | 10.02 | 0.16 | 160 | 10.51 | 0.19 |
| 35 | 80 | 6.62 | 0.37 | 320 | 7.73 | 0.33 | 320 | 7.89 | 0.38 |
| 36 | 160 | 9.007 | 0.1 | 320 | 12.59 | 0.13 | 160 | 9.34 | 0.25 |
| 41 | 80 | 3.415 | 0.6 | 160 | 9.78 | 0.09 | 160 | 10.81 | 0.12 |
| 44 | 160 | 5.805 | 0.04 | 160 | 11.41 | 0.48 | 80 | 10.167 | 0.12 |
| 45 | 160 | 7.22 | 0.27 | 160 | 9.98 | 0.06 | 80 | 10.735 | 0.21 |
| 47 | 160 | 5.97 | 0.05 | 640 | 10.87 | 0.17 | 320 | 10.97 | 0.15 |
| 48 | 160 | 6.798 | 0.2 | 320 | 11.13 | 0.55 | 320 | 13.36 | 0.13 |
| 51 | 80 | 5.09 | 0.12 | 320 | 8.487 | 0.36 | 320 | 8.697 | 0.04 |
| 53 | 320 | 8.71667 | 0.14 | 640 | 11.34 | 0.07 | 160 | 11.51 | 0.24 |
| 65 | 40 | 6.368 | 0.33 | 160 | 7.89 | 0.18 | 160 | 9.65 | 0.06 |

In summary, polyPLA is a simple method quantifying the binding avidity of antibody antigen interactions. While these examples included studies in connection with influenza applications, it will be appreciated that the methods, kits, and tools disclosed herein can also be used in connection other pathogens, including those cannot be propagated in laboratory.

### Non-Human Sample Applications

A large population of genetically and antigenically diverse influenza A viruses (IAVs) is circulating among the swine population, playing an important role in influenza ecology. Swine IAVs not only cause outbreaks among swine, but they can also be transmitted to humans, causing sporadic infections and even pandemic outbreaks. Antigenic characterization of swine IAVs is key to understanding the natural history of these viruses in swine and to selecting strains for effective vaccines. However, influenza outbreaks generally spread rapidly among swine, and the conventional methods for antigenic characterization require virus propagation, a time-consuming process that can significantly reduce the effectiveness of vaccination programs. I developed and validated a rapid, sensitive, and robust method, the polyclonal sera-based proximity ligation assay (polyPLA), to identify antigenic variants of subtype H3N2 swine IAVs. This method utilizes oligonucleotide-conjugated polyclonal antibodies and quantifies antibody-antigen binding affinities by quantitative RT-PCR. Results showed the assay can rapidly detect H3N2 IAVs directly from nasal wash or nasal swab samples collected from laboratory-challenged animals or during influenza surveillance at county fairs. In addition, polyPLA can accurately separate the viruses at two contemporary swine influenza virus (SIV) antigenic clusters (H3SIV-α and H3SIV-β) with a sensitivity of 84.9% and a specificity of 100%. The polyPLA can be routinely used in surveillance programs to detect antigenic variants of influenza viruses and to select vaccine strains for use in controlling and preventing disease in swine. A goal of this study was to develop a specific polyPLA method to quantify the antibody-antigen interaction for swine H3 IAVs. This method would be useful for vaccine strain selection for swine IAVs.

### Materials and Methods

Viruses and serum samples. Seven contemporary (2009-2011) swine H3N2 IAV isolates and their homologous ferret serum samples were chosen to represent the swine influenza virus (SIV) antigenic groups H3a and H3B (TABLE 6); antigenic characterization of these isolates is described elsewhere (Feng et al., 2013). The strain of A/California/04/2009(H1N1) was used as a negative control. NP monoclonal antibody was obtained from BEI Resources (Manassas, Va., USA).

Clinical samples. A total of 120 nasal wash and nasal swab samples were collected for 10 consecutive days post infection (dpi) from 8 feral swine infected with A/swine/Texas/A01104013/2012 (H3N2) and 4 sentinel feral swine. The details for experimental designs and sample collections were available from a prior publication (Sun et al. 2015). Among these samples, 42 were tested with a detectable TCID₅₀, and these samples were used in this study to test the sensitivity and specificity of the proposed polyPLA method. I also tested 81 nasal swab samples that had been collected from swine at the pig exibits at agricultural fairs in Ohio during 2009-2013; 61 of the samples were positive for IAV, using matrix gene-based quantiative RT-PCR (qRT-PCR); and 20 of the samples were negative for IAV. A power analysis (OpenEpi, Version 3) suggested that a sample size of 20 gave 95% probability to detect ±10% with expected specificity (26). Of those 61 IAV-positive samples, 50 were subtype H3 and 11 were subtype H1.

HA and HI assays. HI was performed as previously described (WHO, 2011). In brief, receptor-destroying enzyme (RDE; Denka Seiken Co., Ltd., Tokyo, Japan) was incubated overnight at a 1:3 ratio (vol:vol) with ferret antisera. After incubation, the mixture was heat-inactivated at 56° C. for 30 min and then diluted 1:10 with 1× phosphate-buffered saline (PBS, pH 7.4). The treated ferret anti-serum was then serially diluted in 96-well v-bottom plates with 1× PBS, reacted with 4 HA units of virus, and then incubated for 30 min at 37° C., after which 0.5% turkey RBCs were added to each well and incubated for 30 min at 37° C. The highest dilution in which virus binding to the RBCs was blocked was expressed as the reciprocal HI titer.

polyPLA. IgG was purified from polyclonal serum and monoclonal antibody and labeled separately with 5′ and 3′ TaqMan Prox-Oligos (Thermo Fisher Scientific, Waltham, Mass., USA) for use in a proximity ligation assay, as described elsewhere (Martin et al. 2015). In brief, 5′- and 3′-labeled IgG was diluted (1:10) in assay probe dilution buffer, and 2 μL was added to 2 μL of viruses or 1×PBS (non-protein control [NPC]) and incubated at 37° C. for 1 h. The 96 μL of ligation mixture (0.1 μL of diluted [1:500] ligase, 5 μL of 20× ligation reaction buffer, and 90.9 μL of dH₂O) was added to each incubation product, incubated at 37° C. for 10 min, and then put on ice. Diluted protease was then added to the ligation products and incubated at 37° C. for 10 min and at 95° C. for 5 min and then put on ice. Last, 4.5 μL of protease products was added to 5 μL of TaqMan Protein Assays Fast Master Mix (2×) (Thermo Fisher Scientific, Waltham, Mass., USA) and 0.5 μL 20× Universal PCR Assay (Thermo Fisher Scientific, Waltham, Mass., USA), and quantitative RT-PCR was performed as follows: 95° C. for 20 sec, 40 cycles at 95° C. for 1 sec, and 60° C. for 20 sec. The threshold was set at 0.2, and change in the cycle threshold (ΔC_{T}) were calculated by [average C_{T} (NPC)−average C_{T} (sample)]; quantitative RT-PCR was performed on each sample in triplicate.

Antigenic cartography. The antigenic maps of H3N2 swine IAVs were constructed using AntigenMap (http://sysbio.cvm.msstate.edu/AntigenMap) and data derived from the HI assay or the polyPLA (Cai et al., 2010; Barnett et al., 2012). The data entry with an HI titer of <1:10 or a ΔC_{T} of <3.000 were determined as a low reactor for the data from HI or the polyPLA, respectively.

Data analyses. To make the antigenic properties across the testing antigens (viruses) comparable, I calculated the polyPLA units between virus and antibody as previously described (Martin et al., 2015): polyPLA=a×(polyΔC_{T}−monoΔC_{T})+b, in which a=1.000 and b=10.000, to eliminate negative numbers. A monoΔC_{T} cutoff of <3.000 has been traditionally used to distinguish if virus loads are too low for analyses.

Linear regression analyses were performed using the HI titers of the 7 swine IAV isolates (2 to antigenic clade H3SIV-α and 5 to H3SIV-β) verses their homologous antisera and polyPLA units of these 7 swine IAV isolates verses 3 polyclonal antibodies (1 to H3SIV-α and 2 to H3SIV-β). The 81 nasal swab samples were assessed for monoΔC_{T} and ΔpolyPLA cutoffs by the frequency procedure using SAS 9.4 (SAS Institute Inc., Cary, N.C., USA) to determine sensitivity and specificity for detection of IAVs and antigenic variants with confidence intervals at 95%, receiver operator characteristic area under the curve, and linear regression. The mathematical product of sensitivity x specificity, given the term efficiency, was calculated and graphed for each polyPLA value to provide the probability of correct classification for unknown sample status. Kappa analyses and all descriptive graphs were created using GraphPad Prism version 5.00 for Windows (GraphPad Software, San Diego, Calif., USA).

### RESULTS

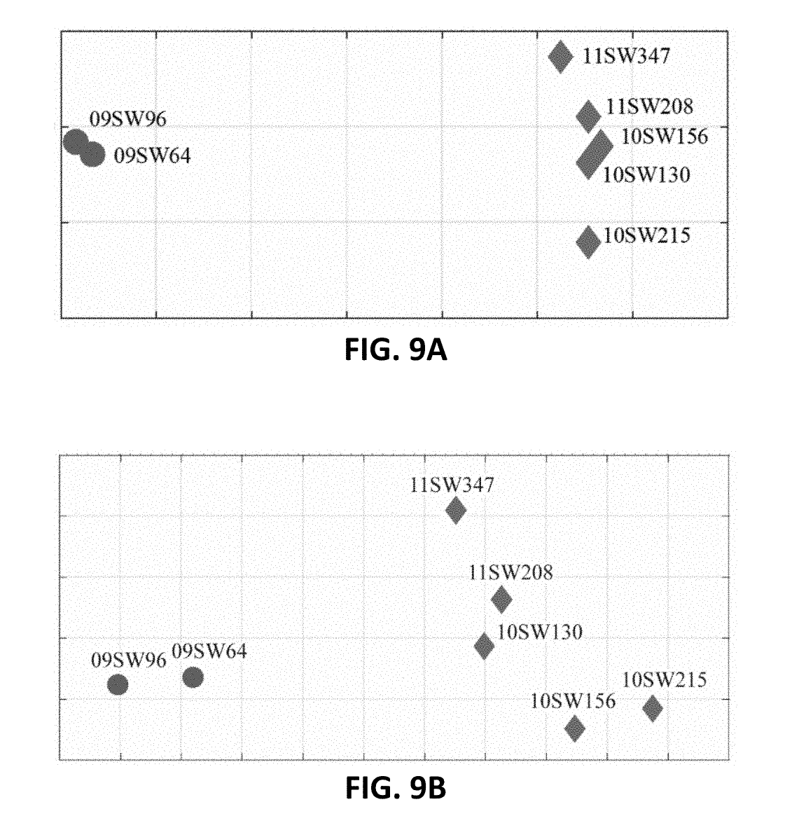

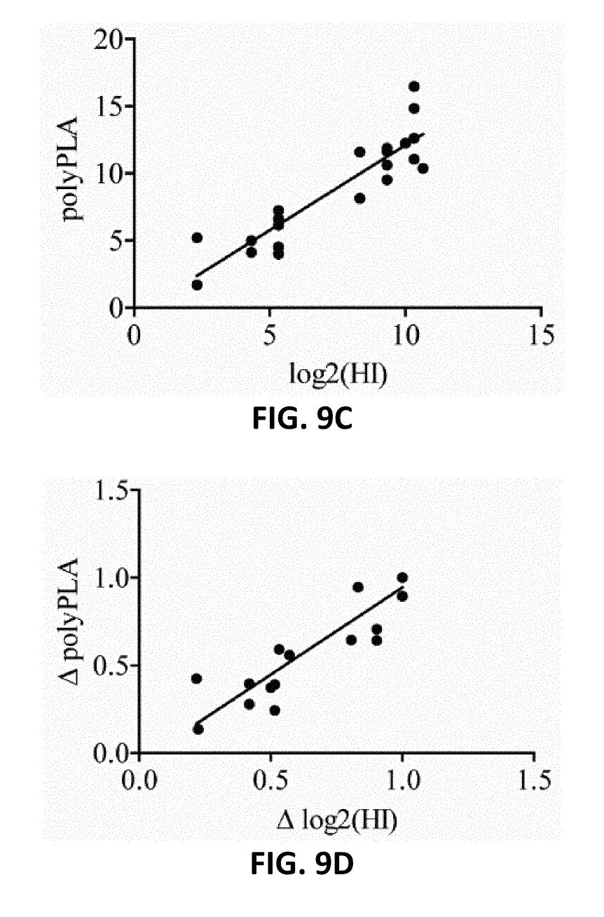

Comparison of HI assay and polyPLA in antigenic characterization of subtype 113 swine IAVs. To assess the effectiveness of the polyPLA, I compared the antigenic data derived from the HI assay and the polyPLA. The seven H3N2 swine IAVs used for testing in the study had cross-reaction titers ranging from <1:10 to 1:1600 (TABLE 6). HI-based antigenic cartography showed that the seven isolates were separated into two antigenic clusters: two isolates from 2009 were in cluster H3 SIV-α, and the five other isolates were in cluster H3SIV-β (FIG. 9A). The results from polyPLA suggested that the titers for these seven testing isolates ranged from 1.710 to 16.474 polyPLA units. In support of the HI cartography-derived data, polyPLA-based cartography also showed that these seven isolates were grouped in two antigenic clusters (FIG. 9A). The average distances between clusters was 5.144 units (±0.149 standard deviation) and 6.268 units (±1.220 standard deviation) in HI and polyPLA cartography, respectively. Correlation association analyses through linear regression showed that the titers between these two types of data had a coefficient of R²=0.8169 (p<0.0001) (FIG. 9C) and that the fold changes in HI titers and polyPLA values had a coefficient of R²=0.8494 (p<0.0001) (FIG. 9D). Similar to HI assay results, polyPLA results suggested that subtype H3N2 swine IAVs did not react with the negative-control H1N1 virus polyclonal antiserum.

TABLE 6
Antigenic characterization of H3N2 swine influenza viruses using
hemagglutination inhibition assay and polyPLA

| | Antigenic Ferret Antiserum^{a} | | | | | | |
|---|---|---|---|---|---|---|---|
| | | 09SW96^{b} | | 10SW215^{b} | | 11SW347^{b} | |
| | | | polyPLA | | polyPLA | | polyPLA |
| Virus | Cluster | HI^{c} | (SD)^{d} | HI^{c} | (SD)^{d} | HI^{c} | (SD)^{d} |
| | | | 10.369 | | 6.142 | | 5.202 |
| A/swine/Ohio/09SW64/2009(H3N2) | H3SIV-α | 1600 | (0.095) | 40 | (0.688) | <10 | (0.360) |
| | | | 11.059 | | 4.525 | | 1.710 |
| A/swine/Ohio/09SW96/2009(H3N2) | H3SIV-α | 1,280 | (0.716) | 40 | (0.709) | <10 | (0.522) |
| | | | 6.623 | | 11.872 | | 10.619 |
| A/swine/Ohio/10SW130/2010(H3N2) | H3SIV-β | 40 | (0.862) | 640 | (0.328) | 640 | (0.281) |
| | | | 4.120 | | 14.847 | | 9.522 |
| A/swine/Ohio/10SW156/2010(H3N2) | H3SIV-β | 20 | (0.372) | 1,280 | (0.363) | 640 | (0.561) |
| | | | 4.011 | | 16.474 | | 11.635 |
| A/swine/Ohio/10SW215/2010(H3N2) | H3SIV-β | 40 | (1.320) | 1,280 | (0.105) | 640 | (0.099) |
| | | | 7.244 | | 11.589 | | 12.258 |
| A/swine/Ohio/11SW208/2011(H3N2) | H3SIV-β | 40 | (0.649) | 320 | (0.461) | 1024 | (0.278) |
| | | | 4.991 | | 8.135 | | 12.631 |
| A/swine/Ohio/11SW347/2011(H3N2) | H3SIV-β | 20 | (0.135) | 320 | (0.441) | 1,280 | (0.387) |
| | | | 6.664 | | 4.828 | | N/A |
| A/California/04/2009(H1N1)^{f} | | 10 | (0.951) | <10 | (0.513) | <10 | (N/A) |

^{a}The viruses were collected from pigs at agricultural fairs in Ohio, USA, 2009-2011 (Feng et al., 2013).
^{b}Homologous titers are in bold.
^{c}095W96, A/swine/Ohio/095W96/2009(H3N2); 105W215, A/swine/Ohio/10SW215/2010(H3N2); 11SW347, A/swine/Ohio/11SW347/2011(H3N2).
^{d}HI, hemagglutination inhibition assay. Titers are an average of the results from two replicates in each experiment.
^{e}polyPLA, polyclonal sera based proximity ligation assay; SD, standard deviation. Values represent the average of three replicate experiments.
^{f}A/California/04/2009 (H1N1) was used as a negative control.

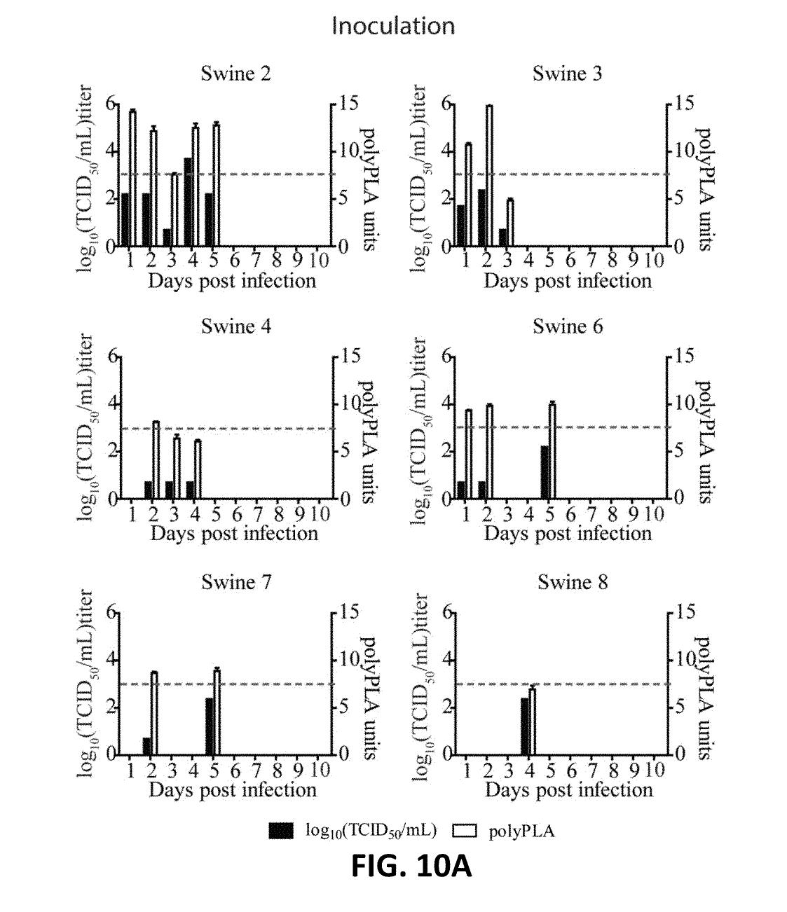

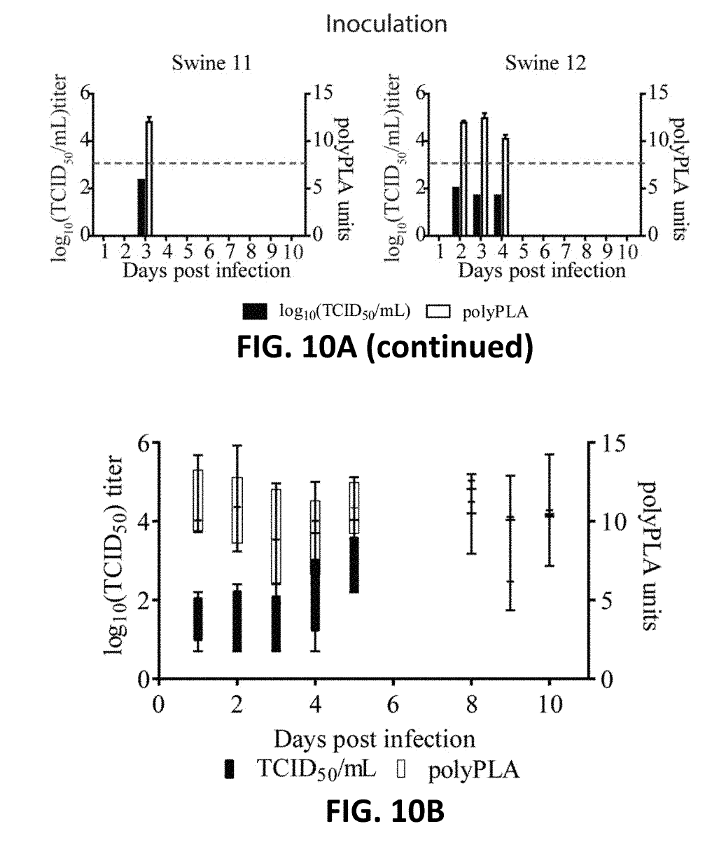

Detection of H3N2 swine IAVs in clinical samples from feral swine. To determine whether polyPLA is sensitive enough to identify H3N2 swine IAVs in clinical samples, I used 42 nasal swab and nasal wash samples from 8 feral swine infected with A/swine/Texas/A01104013/2013(H3N2) (belonging to antigenic clade H3SIV-B) and 4 sentinel feral swine with virus titers up to 5.00×10⁵ TCID₅₀/mL. Of the 42 samples, 34 were IAV-positive (ΔC_{T}≧3.00) using NP monoclonal antibodies. Further analyses using polyclonal H3SIV-β antibodies showed that polyPLA can detect H3N2 swine IAV virus titers of <1,000 TCID₅₀/mL (FIG. 10A); this finding is similar to that for human IAVs which can detect virus titers of <1,000 TCID₅₀/mL, as previously published (Martin et al., 2015). Furthermore, in the animal experiments, polyPLA could detect viral shedding from 1 to 10 days after virus challenge, and titers ranged from 4.35 to 14.83 polyPLA units (FIG. 10B).

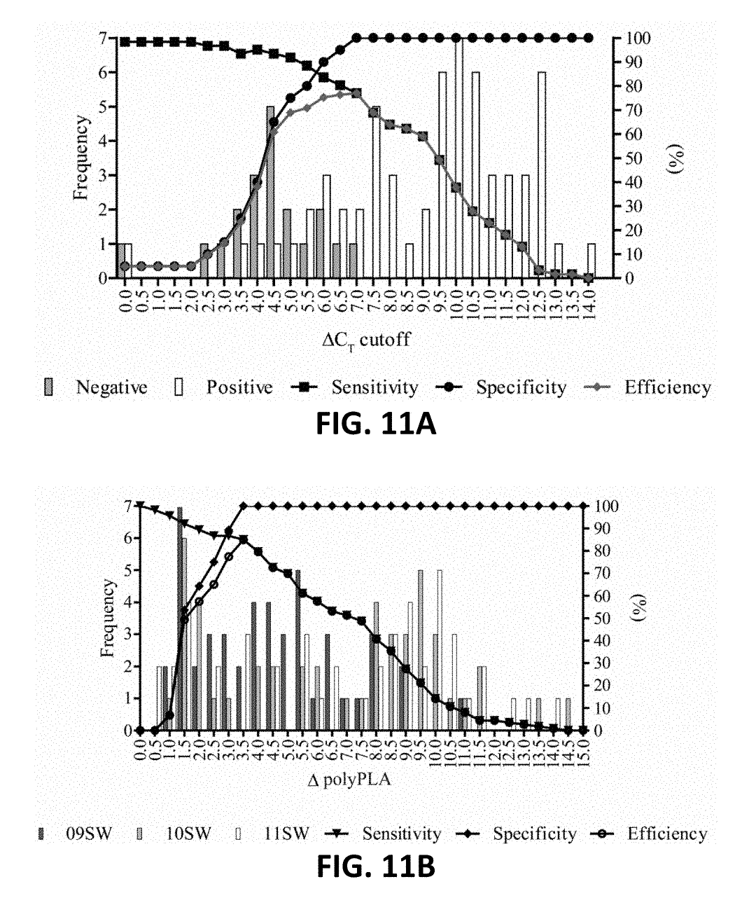

Application of polyPLA in detecting H3N2 swine IAV antigenic variants from clinical samples collected from swine at agricultural fairs. To measure specificity of polyPLA in the clinical setting, I used the assay on 81 samples taken from swine at agricultural fairs. The frequency distribution of ΔC_{T} values for NP monoclonal antibodies for the 61 IAV positive—and 20 IAV negative—clinical samples from domestic swine showed that the greatest efficiency (77.0%) was observed at a ΔC_{T} cutoff of 7.0 (FIG. 11A). Thus, the optimum combination for detecting IAVs in clinical samples is an assay sensitivity of 77.0% (95% CI=64.5%, 86.8%) and specificity of 100.0% (95% CI=83.2%, 100.0%). Accuracy of the polyPLA was measured by the receiver operating characteristic area under the curve, which was 0.90, an excellent test for separating IAV-positive from IAV-negative clinical samples. There was 82.7% overall agreement between qRT-PCR and polyPLA, with a kappa of 62.4% (95% CI=45.8%, 79.0%), which suggests a good strength of agreement. The typical ΔC_{T} cutoff of 3.0 showed that the polyPLA had high sensitivity (96.7%; 95% CI=88.7%, 99.6%) but low specificity (15.0%; 95% CI=3.2%, 37.9%), and 59 true-positive and 17 false-positive samples were detected. At the higher ΔC_{T} cutoff of 7.0, false positives were eliminated, and 47 true positive samples were detected.

To distinguish between antigenic group H3SIV-α and antigenic group H3SIV-β viruses, I calculated the frequency distribution of ΔpolyPLA values for H3SIV-α and H3SIV-β polyclonal antibodies for the 47 IAV-positive clinical samples from domestic swine. At the ΔpolyPLA threshold of 3.5, the greatest efficiency was observed at 85.0%, with a sensitivity of 85.0% (95% CI=77.0%, 91.0%) and specificity of 100.0% (95% CI=87.7%, 100.0%) (FIG. 11B). Correlation association analyses through linear regression showed the fold increment titers from homologous virus isolates and fold increment in polyPLA values had a coefficient of R²=0.88 (p<0.0001). An 8-fold increment in HI titer was correlated with a 3.26-fold increment in polyPLA units. polyPLA was able to distinguish between the two swine IAV H3 antigenic groups with complete agreement: 10 samples were H3SIV-a-positive (sensitivity 95% CI=69.2%; 100%), and 33 were H3SIV-B-positive (sensitivity 95% CI=89.4%; 100%); 4 were negative to both polyclonal antibodies because they were previously identified as H1 qRT-PCR-positive.

Effectiveness of polyPLA in detecting H1 swine IAV antigenic variants. To evaluate whether polyPLA was effective in identifying antigenic variants for subtype H1 IAVs, cross-activities were measured using both polyPLA and HI assays between the CA/04 polyclonal serum and a panel of H1N1 isolates, which belong to 8 antigenically distinct clades, including clade H1α, H1β, H1γ, H1γ1, H1γ2, H1δ1, H1δ2, and A(H1N1)pdm09. Results from polyPLA showed that CA/04 polyclonal serum cross-reacted with the homologous virus CA/04 and A/swine/Iowa/8/2013(H1N1) (both to clade A(H1N1)pdm09) with two highest polyPLA units, 13.83 and 15.87, respectively; this serum cross reacted to A/swine/Nebraska/A01240348/2011(H1N1) (H1β) with 12.02 polyPLA unit, to A/swine/Indiana/13TOSU1154/2013(H1N1) (H1γ) with 11.01 polyPLA unit, to A/swine/Indiana/13TOSU0832/2013(H1N1) (H1γ1) with 10.22 polyPLA unit. The polyPLA values for the other four viruses (H1α, H1γ2, H1δ1, and H1δ2) were less than 10.00 (Supplementary Table 3). Such results were consistent with the corresponding HI titers, validating that this method is effective in antigenic characterization of H1 viruses. Furthermore, I performed polyPLA assays using CA/04 serum against the 47 IAV-positive clinical samples. Results showed that CA/04 serum did not cross-react with 43 H3 IAV positive samples but did to 4 H1 IAV positive samples to different extents. Among these 4 H1 viruses, 2 were sequenced, 1 genetically belong to Hly and the other one to H1δ1 (TABLE 7).

TABLE 7
Antigenic differences in subtype H1 isolates and clinical samples from
swine, using the HI assay and the polyPLA^{a} with polyclonal antibody against
A/California/04/2009(H1N1).

| Clinical | | | HI^{e} | ΔCt^{f} | ΔCt | | polyPLA^{g} | |
|---|---|---|---|---|---|---|---|---|
| Sample ID^{b} | Isolate | Genetic cluster^{c} | CA/04^{d} | MAB | CA/04 | SD | CA/04 | SD |
| N/A | A/California/04/2009(H1N1) | A(H1N1)pdm09 | 160 | 3.27 | 7.10 | 0.17 | 13.83 | 0.17 |
| N/A | A/swine/Iowa/8/2013(H1N1) | A(H1N1)pdm09 | 320 | 3.15 | 9.02 | 1.09 | 15.87 | 1.09 |
| N/A | A/swine/Minnesota/A01394082/2013(H1N1) | H1α | <10 | 3.32 | 2.67 | 0.10 | ND | ND |
| N/A | A/swine/Nebraska/A01240348/2011(H1N1) | H1β | 640 | 7.83 | 9.84 | 0.85 | 12.02 | 0.85 |
| N/A | A/swine/Indiana/13TOSU1154/2013(H1N1) | H1γ | 320 | 3.47 | 4.48 | 0.17 | 11.01 | 0.17 |
| N/A | A/swine/Indiana/13TOSU0832/2013(H1N1) | H1γ1 | 40 | 3.06 | 3.28 | 0.21 | 10.22 | 0.21 |
| N/A | A/swine/Illinois/A01076767/2010(H1N1) | H1γ2 | <10 | 6.24 | 3.43 | 0.28 | 7.19 | 0.28 |
| N/A | A/swine/Iowa/15/2013(H1N1) | H1δ1 | <10 | 4.20 | 3.69 | 0.25 | 9.49 | 0.25 |
| N/A | A/swine/Iowa/18/2013(H1N1) | H1δ2 | <10 | 3.07 | 2.54 | 0.24 | ND | ND |
| TOSU56 | A/swine/Ohio/11SW174/2011(H1N2) | H1δ1 | ND | 9.38 | 5.58 | 0.25 | 6.2 | 0.25 |
| TOSU58 | A/swine/Kentucky/12TOSU1053/2012(H1N2) | H1δ1 | ND | 6.22 | 3.28 | 0.48 | 7.06 | 0.48 |
| TOSU55 | A/swine/Ohio/11SW129/2011(H1N2) | H1δ1 | ND | 5.84 | 5.47 | 1.12 | 9.63 | 1.12 |
| TOSU59 | A/swine/Kentucky/12TOSU1054/2012(H1N2) | H1δ1 | ND | 5.7 | 3.09 | 0.83 | 7.39 | 0.83 |
| TOSU54 | A/swine/Ohio/11SW128/2011(H1N2) | H1δ1 | ND | 5.48 | 5.37 | 1.07 | 9.89 | 1.07 |
| TOSU57 | A/swine/Ohio/11SW192/2011(H1N2) | H1δ1 | ND | <3.0 | ND | ND | ND | ND |
| TOSU51 | A/swine/Indiana/13TOSU486/2013(H1N1) | H1γ | ND | 7.02 | 4.04 | 0.97 | 7.02 | 0.97 |
| TOSU61 | A/swine/Ohio/12TOSU45/2012(H1N1) | H1γ | ND | 5.08 | 2.22 | 1.68 | ND | ND |
| TOSU52 | N/A | N/A | ND | 9.34 | 3.9 | 0.51 | 4.56 | 0.51 |
| TOSU53 | N/A | N/A | ND | 10.04 | 5.14 | 0.58 | 5.1 | 0.58 |
| TOSU60 | N/A | N/A | ND | 4.55 | 0.53 | 0.12 | 5.98 | 0.12 |

^{a}HI, hemagglutination inhibition; polyPLA, polyclonal sera-based proximity ligation assay; SD, standard deviation.
^{b}Clinical samples from swine at Ohio, USA, agricultural fairs;
^{c}Genetic cluster defined by the Influenza Research Database Swine H1 Classification Tool.
^{d}CA/04, A/California/04/2009 (H1N1).
^{e}Hemagglutination inhibition virus titers in H1 viral isolates. Each assay was performed in three independent experiments, each of which with two replicates.
^{f}The numbers in bold denote homologous titers, and the numbers underlined denotes those samples as IAV positive using polyPLA with a ΔC_{T} cutoff of 7.0.
^{g}polyPLA values of swine clinical samples and viral isolates performed in triplicate.

### DISCUSSION

In this study, a polyPLA assay was used to detect antigenic variants of contemporary subtype H3 IAVs in swine in the United States. This assay was validated to differentiate viruses in antigenic cluster H3SIV-α from those in H3SIV-β directly in clinical samples, such as swine nasal swab or nasal wash samples. With the optimized ΔpolyPLA unit of 3.500, this assay can detect antigenic variants with a specificity of 100% and a sensitivity of 84.9% in samples collected during influenza surveillance. Thus, the polyPLA is specific and sensitive enough to be used for vaccine strain selection for swine IAVs. Because it does not require virus isolation, this assay shortens the time needed for antigenic characterization using conventional methods (3-5 days) to only a few hours after specimen collection and, therefore, can increase effectiveness of vaccination programs on swine farm operations and at agricultural fairs. In addition, because it is developed based on a qRT-PCR platform, polyPLA can be used for high-throughput screening and for clinical diagnosis in most laboratories.

The HI assay is used routinely in influenza antigenic characterization because of its ease of access and its capacity for medium-throughput screening. The principle of HI is based on the competition of the glycan receptors on animal RBCs and antibody against surface glycoproteins, especially hemagglutinins of IAVs. Thus, antigenic characterization results can be affected not only by changes at antibody binding sites, but also by the source of the RBCs and the variation of receptor binding sites. In addition, HI requires a large amount of virus particles, so, in general, it is necessary to recover and propagate viruses using cells or chicken eggs, which can lead to unwanted adaptive mutations, especially those at the receptor binding sites of viral hemagglutinin proteins. These mutations can skew HI data and even cause loss of binding affinity to some RBCs (Medeiros et al., 2001; Nobusawa et al., 2000). In addition, because no standard RBCs are used in HI assays, the variation in the binding affinities of IAVs to different sources of RBCs make it difficult to interpret those results across HI assays using different RBCs. Unlike the HI assay, polyPLA does not use RBCs and is not affected by variations in receptor binding sites. Instead, polyPLA utilizes oligonucleotide-labeled antibodies and quantifies the binding affinities between antibody and antigen through qRT-PCR. More strikingly, this method can be applied directly in clinical samples and can minimize biases due to virus adaptation in virus isolation. Results showed that polyPLA values are similar to those in HI assays, although the scale of fold increments are different. In this study, both HI and polyPLA clearly separated swine IAVs in antigenic cluster H3SIV-α from those in antigenic cluster H3SIV-β. Furthermore, an eight-fold increment in HI titer was approximately equal to a 3.256-fold increment in polyPLA.

Compared with clinical samples from laboratory animals, samples derived from the field could be complicated with high background in qRT-PCR due to low quantities of virus analyte, inappropriately collected/handled/transported specimens, presence of viral inhibitor, and/or presence of proteins from other viruses or bacterial pathogens (Petric et al., 2006). Nevertheless, for vaccine strain selection in the clinical setting, it is critical to use assays with 100% specificity and relatively high sensitivity. Relatively low assay sensitivity can be overcome by using a larger number of samples in the assays; in general, the availability of multiple samples from swine herds will not be an issue, especially during an outbreak. Based on the 81 samples I tested, polyPLA has a sensitivity of 77.0% (95% CI of 55.7%, 80.1%) and specificity of 100.0% (95% CI of 83.2%, 100.0%) when the ΔCt cutoff is set at 7.0 (FIG. 11A).

The number of polyPLAs conducted in experiments can be reduced if the IAV subtype in samples is known prior to testing. Thus, the performance of polyPLA can be maximized and the cost can be reduced if the assay is coupled with subtype-specific IAV antigen assays, such as qRT-PCR. The recommended procedure for polyPLA application includes three steps: 1) determine whether the testing sample is IAV-positive by using a matrix gene-based qRT-PCR (WHO, 2014) rapid influenza antigen detection test (Anonymous, 1999) or an influenza test strip; 2) use qRT-PCR to determine the subtype of IAV in the sample; and 3) perform antigenic characterization by using subtype-specific polyPLA. As with the conventional methods for antigenic characterization, polyPLA can be used with a panel of reference sera to quantify antigenic diversity among the viruses; thus, polyPLA is useful for antigenic characterization of IAVs and other pathogens, as previously demonstrated above.

All publications, patents, and patent applications mentioned in this specification are herein incorporated by reference, as indicated herein, to the same extent as if each individual publication, patent, or patent application was specifically and individually indicated to be incorporated by reference, including the references set forth in the following list:
Anonymous 1999. The Medical Letter. 1999. Rapid diagnostic tests for influenza. Med Lett Drugs Ther 41:121-122.
2010. Estimates of deaths associated with seasonal influenza—United States, 1976-2007. MMWR Morb Mortal Wkly Rep 59, 1057-1062.
WHO 2011. World Health Organization. Manual for the laboratory diagnosis and virological surveillance of influenza WHO Global Influenza Surveillance Network, WHO, Geneva, Switzerland.
2013. Update of WHO biosafety risk assessment and guidelines for the production and quality control of human influenza vaccines against avian influenza A(H7N9) virus.
WHO 2014. World Health Organization. (revised May 2015: Annex 2.E and Annex 2.D). WHO information for molecular diagnosis of influenza virus in humans—update. WHO, Geneva, Switzerland.
Ampofo, W. K., Baylor, N., Cobey, S., Cox, N. J., Daves, S., Edwards, S., Ferguson, N., Grohmann, G., Hay, A., Katz, J., Kullabutr, K., Lambert, L., Levandowski, R., Mishra, A. C., Monto, A., Siqueira, M., Tashiro, M., Waddell, A. L., Wairagkar, N., Wood, J., Zambon, M., Zhang, W., 2012. Improving influenza vaccine virus selection: report of a WHO informal consultation held at WHO headquarters, Geneva, Switzerland, 14-16 Jun. 2010. Influenza Other Respi Viruses 6, 142-152.
Azzi, A., Bartolomei-Corsi, O., Zakrzewska, K., Corcoran, T., Newman, R., Robertson, J. S., Yates, P., Oxford, J. S., 1993. The haemagglutinins of influenza A (H1N1) viruses in the ‘0’ or ‘D’ phases exhibit biological and antigenic differences. Epidemiol Infect 111, 135-142.
Baccam, P., Beauchemin, C., Macken, C. A., Hayden, F. G., Perelson, A. S., 2006. Kinetics of influenza A virus infection in humans. J Virol 80, 7590-7599.
Barnett J L, Yang J, Cai Z, Zhang T, Wan X F. 2012. AntigenMap 3D: an online antigenic cartography resource. Bioinformatics 28:1292-1293.
Cai Z, Zhang T, Wan X F. 2010. A computational framework for influenza antigenic cartography. PLoS Comput Biol 6:e1000949.
Cox, N. J., Bender, C. A., 1995. The molecular epidemiology of influenza viruses. Seminars in Virology 6, 359-370.
Edgar, R. C., 2004. MUSCLE: multiple sequence alignment with high accuracy and high throughput. Nucleic Acids Res 32, 1792-1797.
Feng Z, Gomez J, Bowman A S, Ye J, Long L P, Nelson S W, Yang J, Martin B, Jia K, Nolting J M, Cunningham F, Cardona C, Zhang J, Yoon K J, Slemons R D, Wan X F. 2013. Antigenic characterization of H3N2 influenza A viruses from Ohio agricultural fairs. J Virol 87:7655-7667.
Fitch, W. M., Bush, R. M., Bender, C. A., Cox, N. J., 1997. Long term trends in the evolution of H(3) HA1 human influenza type A. Proc Natl Acad Sci USA 94, 7712-7718.
Fredriksson, S., Dixon, W., Ji, H., Koong, A. C., Mindrinos, M., Davis, R. W., 2007. Multiplexed protein detection by proximity ligation for cancer biomarker validation. Nature methods 4, 327-329.
Gambaryan, A. S., Robertson, J. S., Matrosovich, M. N., 1999. Effects of egg-adaptation on the receptor-binding properties of human influenza A and B viruses. Virology 258, 232-239.
Gerdil, C., 2003. The annual production cycle for influenza vaccine. Vaccine 21, 1776-1779.
Grund, S., Adams, O., Wahlisch, S., Schweiger, B., 2011. Comparison of hemagglutination inhibition assay, an ELISA-based micro-neutralization assay and colorimetric microneutralization assay to detect antibody responses to vaccination against influenza A H1N1 2009 virus. J Virol Methods 171, 369-373.
Harper, S. A., Fukuda, K., Uyeki, T. M., Cox, N. J., Bridges, C. B., 2004. Prevention and control of influenza: recommendations of the Advisory Committee on Immunization Practices (ACIP). MMWR Recomm Rep 53, 1-40.
Hirst, G. K., 1941. The Agglutination of Red Cells by Allantoic Fluid of Chick Embryos Infected with Influenza Virus. Science 94, 22-23.
Katz, J. M., Naeve, C. W., Webster, R. G., 1987. Host cell-mediated variation in H3N2 influenza viruses. Virology 156, 386-395.
Lindstrom, S., Endo, A., Sugita, S., Pecoraro, M., Hiromoto, Y., Kamada, M., Takahashi, T., Nerome, K., 1998. Phylogenetic analyses of the matrix and non-structural genes of equine influenza viruses. Arch Virol 143, 1585-1598.
Lindstrom, S., Sugita, S., Endo, A., Ishida, M., Huang, P., Xi, S. H., Nerome, K., 1996. Evolutionary characterization of recent human H3N2 influenza A isolates from Japan and China: novel changes in the receptor binding domain. Arch Virol 141, 1349-1355.
Martin B E, Jia K, Sun H, Ye J, Hall C, Ware D, Wan X F. 2015. Detection of influenza antigenic variants directly from clinical samples using polyclonal antibody based proximity ligation assays. Virology 476:151-158.
Medeiros, R., Escriou, N., Naffakh, N., Manuguerra, J. C., van der Werf, S., 2001. Hemagglutinin residues of recent human A(H3N2) influenza viruses that contribute to the inability to agglutinate chicken erythrocytes. Virology 289, 74-85.
Mori, S. I., Nagashima, M., Sasaki, Y., Mori, K., Tabei, Y., Yoshida, Y., Yamazaki, K., Hirata, I., Sekine, H., Ito, T., Suzuki, S., 1999. A novel amino acid substitution at the receptor-binding site on the hernagglutinin of H3N2 influenza A viruses isolated from 6 cases with acute encephalopathy during the 1997-1998 season in Tokyo. Arch Virol 144, 147-155.
Morishita, T., Kobayashi, S., Miyake, T., Ishihara, Y., Nakajima, S., Nakajima, K., 1993. Hostspecific hemagglutination of influenza A (H1N1) virus. Microbiol Immunol 37, 661-665.
Morishita, T., Nobusawa, E., Nakajima, K., Nakajima, S., 1996. Studies on the molecular basis for loss of the ability of recent influenza A (H1N1) virus strains to agglutinate chicken erythrocytes. J Gen Virol 77 (Pt 10), 2499-2506.
Nobusawa, E., Ishihara, H., Morishita, T., Sato, K., Nakajima, K., 2000. Change in receptorbinding specificity of recent human influenza A viruses (H3N2): a single amino acid change in hemagglutinin altered its recognition of sialyloligosaccharides. Virology 278, 587-596.
Nobusawa, E., Nakajima, K., 1988. Amino acid substitution at position 226 of the hemagglutinin molecule of influenza (H1N1) virus affects receptor binding activity but not fusion activity. Virology 167, 8-14.
Oh, D. Y., Barr, I. G., Mosse, J. A., Laurie, K. L., 2008. MDCK-SIAT1 cells show improved isolation rates for recent human influenza viruses compared to conventional MDCK cells. J Clin Microbiol 46, 2189-2194.
Patterson, S., Oxford, J. S., 1986. Analysis of antigenic determinants on internal and external proteins of influenza virus and identification of antigenic subpopulations of virions in recent field isolates using monoclonal antibodies and immunogold labelling. Arch Virol 88, 189-202.
Petric M, Comanor L, Petti C A. 2006. Role of the laboratory in diagnosis of influenza during seasonal epidemics and potential pandemics. J Infect Dis 194 Suppl 2:S98-110.
Schlingemann, J., Leijon, M., Yacoub, A., Schlingemann, H., Zohari, S., Matyi-Toth, A., Kiss, I., Holmquist, G., Nordengrahn, A., Landegren, U., Ekstrom, B., Belak, S., 2010. Novel means of viral antigen identification: improved detection of avian influenza viruses by proximity ligation. Journal of virological methods 163, 116-122.
Services, W.C.C.f.R.a.R.o.I.U.S.D.o.H.a.H., 1982. Concepts and procedures for laboratory-based influenza surveillance.
Skowronski, D. M., Masaro, C., Kwindt, T. L., Mak, A., Petrie, M., Li, Y., Sebastian, R., Chong, M., Tam, T., De Serres, G., 2007. Estimating vaccine effectiveness against laboratory-confirmed influenza using a sentinel physician network: results from the 2005-2006 season of dual A and B vaccine mismatch in Canada. Vaccine 25, 2842-2851.
Sun, H., Yang, J., Zhang, T., Long, L. P., Jia, K., Yang, G., Webby, R., Wan, X. -F., 2013. Inferring influenza virus antigenicity using sequence data. mBio 4, 4.
Sun H, Cunningham FL, Harris J, Xu Y, Long L P, Hanson-Dorr K, Baroch J A, Fioranelli P, Lutman M , Li T, Pedersen K, Schmit B S, Cooley J, Lin X, Jarman R G, DeLiberto T J, Wan X F. 2015. Dynamics of virus shedding and antibody responses in influenza A virus-infected feral swine. J Gen Virol 96:2569-2578.
Swofford, D. L., 1998. PAUP*: Phylogenic analysis using Parsimony. Sinauer, Sunderland, Mass.
Wan, X. F., Nguyen, T., Davis, C. T., Smith, C. B., Zhao, Z. M., Carrel, M., Inui, K., Do, H. T., Mai, D. T., Jadhao, S., Balish, A., Shu, B., Luo, F., Emch, M., Matsuoka, Y., Lindstrom, S. E., Cox, N. J., Nguyen, C. V., Klimov, A., Donis, R. O., 2008. Evolution of highly pathogenic H5N1 avian influenza viruses in Vietnam between 2001 and 2007. PLoS One 3, e3462.
Webster, R. G., Laver, W. G., Air, G. M., Schild, G. C., 1982. Molecular mechanisms of variation in influenza viruses. Nature 296, 115-121.
Wilson, I. A., Cox, N. J., 1990. Structural basis of immune recognition of influenza virus hemagglutinin. Annu Rev Immunol 8, 737-771.
Zwickl, D. J., 2006. Genetic algorithm approaches for the phylogenetic analysis of large biological sequence datasets under the maximum likelihood criterion. The University of Texas Austin.
Schlingemann, et al., 2010. Novel means of viral antigen identification: Improved detection of avian influenza viruses by proximity ligation. J. Virol. Methds. 163(1): 116-22.
Gutafsdottir et al. 2006. Detection of Individual Microbial Pathogens by Proximity Ligation. Clinical Chemistry 52:61152-1160.
Lundberg, et al., 2011. Multiplexed Hologeneous Proximity Ligation Assays for High-throughput Protein Biomarker Research in Serological Material. Molecular & Cellular Proteomics, (4):M110.004978.
U.S. Patent Application Publication No. 2008/0293051.
U.S. Patent Application Publication No. 2008/0008997.
U.S. Patent Application Publication No. 2008/0090238.
U.S. Patent Application Publication No. 2010/0240101.

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition or step being referred to is an optional (not required) feature of the invention.

Although any methods, devices, and materials similar or equivalent to those described herein can be used in the practice or testing of the presently-disclosed subject matter while remaining within the spirit and scope of the invention, representative methods, devices, and materials are described herein. It will be apparent to one of ordinary skill in the art that methods, devices, device elements, materials, procedures, and techniques other than those specifically described herein can be applied to the practice of the invention as broadly disclosed herein without resort to undue experimentation. All art-known functional equivalents of methods, devices, device elements, materials, procedures and techniques described herein are intended to be encompassed by this invention. Whenever a range is disclosed, all sub-ranges and individual values are intended to be encompassed. This invention is not to be limited by the embodiments disclosed, including any shown in the drawings or exemplified in the specification, which are given by way of example and not of limitation.

The systems and methods of the present disclosure, including components thereof, can comprise, consist of, or consist essentially of the essential elements and limitations of the embodiments described herein, as well as any additional or optional components or limitations described herein or otherwise useful.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself For example, if the value “10” is disclosed, then “about 10” is also disclosed. It is also understood that each unit between two particular units are also disclosed. For example, if 10 and 15 are disclosed, then 11, 12, 13, and 14 are also disclosed.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

All patents, patent applications, published applications, publications, GenBank sequences, databases, websites, and other published materials referred to throughout the entire disclosure herein, unless noted otherwise, are incorporated by reference in their entirety, to the extent each reference is at least partially not inconsistent with the disclosure in the present application (for example, a reference that is partially inconsistent is incorporated by reference except for the partially inconsistent portion of the reference). In certain instances, nucleotides and polypeptides disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application. Where reference is made to a URL or other such identifier, address, or statement of availability, it understood that such identifiers can change and particular information on the Internet can come and go, but equivalent information can be found by searching the Internet. Reference thereto evidences the availability and public dissemination of such information.

1. A method for detecting antigenic variants of a pathogen comprising:
incubating a labeled polyclonal antiserum with a sample containing the pathogen; and
quantifying antiserum-pathogen binding avidity, wherein the quantifying step includes a further step of conducting a proximity ligation assay coupled to quantitative PCR with the incubated labeled polyclonal antiserum.

2. The method of claim 1 further comprising steps of purifying a polyclonal antiserum prior and labeling the purified polyclonal antiserum.

3. The method of claim 2, wherein the purifying step includes chromatographic purification of IgG from the polyclonal antiserum.

4. The method of claim 2, wherein the labeling step includes labeling the purified polyclonal antiserum with a pair of oligonucleotides comprising a 5′ primer and a 3′ primer.

5. The method of claim 4, wherein the pair of oligonucleotides is a pair of sodium azide-linked oligonucleotides and wherein the purified polyclonal antiserum is further pre-labeled with biotin prior to labeling with the pair of oligonucleotides

6. The method of claim 5, wherein the proximate ligation assay includes a step of the sodium azide-linked 5′ primer and the sodium azide-linked 3′ primer annealing to an included connector oligonucleotide and a step of ligating with an added ligase enzyme.

7. The method of claim 1, wherein the quantifying step further includes a step of normalizing the labeled polyclonal antiserum proximity ligation assay.

8. The method of claim 7, wherein the normalizing step includes the steps of lysing the pathogen to release an antigen comprising a conserved epitope.

9. The method of claim 8, wherein the lysing step includes treating the pathogen sample with a lysis buffer.

10. The method of claim 8, wherein the lysing step includes treating the pathogen sample with consecutive freeze-thaw cycles.

11. The method of claim 1, wherein the pathogen is a bacterium.

12. The method of claim 1, wherein the pathogen is a virus.

13. The method of claim 12, wherein the virus is an influenza virus.

14. A method of detecting antigenic variation comprising: providing purified polyclonal antiserum; providing at least one monoclonal antibody of interest; providing a proximity ligation assay; providing at least two oligonucleotide assay proximity probes comprising a 5′ primer and a 3′ primer; providing a linker molecule for labeling the purified polyclonal antiserum and the monoclonal antibody of interest; and providing instructions for performing the proximity ligation assay.

15. The method of claim 14, wherein performing the proximity ligation assay includes steps of labeling the purified polyclonal antiserum and the at least one monoclonal antibody of interest with biotin; preparing the at least two oligonucleotide assay proximity probes for each biotinylated antiserum and antibody by forming a plurality of a first mixture for the biotinylated antiserum and antibody; incubating the first mixture samples of the biotinylated antiserum and antibody with the at least two oligonucleotide assay proximity probes for about one hour; diluting the first mixture samples and adding a diluted lyzed pathogen to form a plurality of second mixtures and incubating for about one hour; initializing a ligation reaction for each of the second mixtures; reacting the diluted pathogen with the labeled purified polyclonal antiserum to determine binding avidity between the pathogen and labeled purified polyclonal antiserum; and utilizing a quantitative polymerase chain reaction platform to determine assay results, wherein the proximity ligation assay detects antigenic variants using the purified polyclonal antiserum.

16. The method of claim 14, wherein providing a pair of oligonucleotide assay proximity probes comprises providing instructions for preparing the at least two assay proximity probes.

17. The method of claim 14, wherein the diluted pathogen component is replaced with a non-diluted pathogen sample for direct analysis.

18. A kit for detecting a pathogenic antigenic variation comprising: a purified polyclonal antiserum prepared by inoculating an appropriate host with a pathogen; a monoclonal antibody with binding affinity to a conserved epitope of the pathogen; and a set of reagents for performing a quantitative polymerase chain reaction coupled to a proximity ligation assay.

19. The kit of claim 18, wherein the pathogen is an influenza virus.

20. The kit of claim 18, further comprising instructions for performing a proximity ligation assay with the provided reagents.
